(19) United States
(12) Patent Application Publication
(10) Pub. No.: US 2006/0254601 A1
(43) Pub. Date: Nov. 16, 2006

(54) Difficulty evaluating method, risk diagnosing method, characteristic evaluating method, task allocating method, and task improving method

(75) Inventors: Yoshiharu Matsumura, Kanagawa (JP); Masaya Kaneko, Kanagawa (JP); Osamu Kikuchi, Tokyo (JP); Takeshi Yoshikawa, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/401,904

(22) Filed: Apr. 12, 2006

(30) Foreign Application Priority Data
Apr. 12, 2005 (JP) 2005-114958
Apr. 20, 2005 (JP) 2005-122717

(51) Int. Cl. A61B 19/00

(52) U.S. Cl. 128/898

(57) ABSTRACT

A producing sequence is constituted by a plurality of tasks. To manage production, a task difficulty level of a task within the producing sequence is evaluated. INPUT, PROCESS and OUTPUT skill categories are determined by categorizing a skill required for human resource in charge of the task according to cognitive science. The INPUT skill category is related to cognition of outer field information or acquisition of cognitive information. The PROCESS skill category is related to thinking or decision. The OUTPUT skill category is related to behavior or action. The task is evaluated with the INPUT, PROCESS and OUTPUT skill categories, to obtain respectively difficulty ranks. The task difficulty level is determined by processing the difficulty ranks in combination according to overall processing. Also, a risk diagnosis is provided, in which an error influence level of a predictable error of a task within a producing sequence is assessed.

### BACKGROUND OF THE INVENTION

1. Field of the Invention

The present invention relates to a difficulty evaluating method, risk diagnosing method, characteristic evaluating method, task allocating method, and task improving method. More particularly, the present invention relates to a difficulty evaluating method, risk diagnosing method, characteristic evaluating method, task allocating method, and task improving method, with which production in a producing system can be managed in an optimized manner.

2. Description of the Related Art

In a producing line, automated machinery is installed to manufacture industrial products. Workers operate the machinery, and if an error or failure occurs, handle the error in order to recover the normal operation. Thus, productivity of a producing sequence is ensured as well as the product quality. There has been a recent requirement of utilization of leased workers in combination with employees of a manufacturer. Factors of the background of this include a decrease in skilled workers, and desire for reducing the manufacturing cost, so outsourcing is developed in the producing line.

The producing line is constituted by a plurality of producing sequences, each of which includes plural tasks. In general, an administrator of the producing line considers difficulty of tasks, importance, constraint of available time, skill of workers and the like according to his or her experience of managing production, so that the administrator allocates the tasks to the workers. Tasks are allocated to the leased workers in a similar manner. There is a tendency in that tasks with a low level of the difficulty or importance, or with little constraint of available time are allocated to leased workers as viewed in a relative point of view.

However, allocation of tasks in the known technique does not have definite standards, and is determined by the administrator case by case. A task that should be allocated to the employees may be allocated to leased workers. In turn, a task that should be allocated to the leased workers may be allocated to the employees. Such unsuitable allocation is likely to influence productivity in view of utilizing human resource. Considerable influence in the product quality may occur due to a rise in a ratio of occurrence of errors in operation.

Various documents, such as U.S. Pat. No. 6,249,715 (JP-A 10-261122) and JP-A 2004-139515, suggest evaluation of the producing sequence and optimization of allocation of tasks for the purpose of maximizing efficiency in the producing line. The difficulty and a degree of learning of tasks are analyzed according to working time or the like, and considered in the allocation. Also, the producing sequence is evaluated according to load of working in the tasks in view of physical and mental factors. However, no known document disclose selective allocation between the employees and leased workers.

It is necessary in the producing sequence with various devices for workers to understand the present status of machinery, and monitor product quality of obtained products for its acceptability. However, the method of U.S. Pat. No.6,249,715 (JP-A 10-261122) does not manage errors promptly, because the present status is monitored only on the basis of working time. The method of JP-A 2004-139515 evaluates load of working to the workers. However, no portion of the document suggests evaluation regarding tasks objectively in a quantitative manner.

### SUMMARY OF THE INVENTION

In view of the foregoing problems, an object of the present invention is to provide a difficulty evaluating method, risk diagnosing method, characteristic evaluating method, task allocating method, and task improving method, with which production in a producing system can be managed in an optimized manner.

In order to achieve the above and other objects and advantages of this invention, a difficulty evaluating method of evaluating a task difficulty level of a task within a producing sequence is provided. In the difficulty evaluating method, a skill required for human resource to perform the task according to cognitive science is categorized, to determine at least first, second and third skill categories, the first skill category being related to cognition of outer field information or acquisition of cognitive information, the second skill category being related to thinking or decision, and the third skill category being related to behavior or action. The task with the first, second and third skill categories are evaluated, to obtain respectively difficulty ranks. The task difficulty level is determined by processing the difficulty ranks in combination according to overall processing.

Plural factors are provided in each of the first, second and third skill categories. Factors of the first skill category among the factors have an evaluating factor of knowledge with sufficient quality or quantity for correctly recognizing a status, and an evaluating factor of cognition with degree of necessity of sensory organs for correctly recognizing a status. Factors of the second skill category among the factors have an evaluating factor of knowledge with sufficient quality or quantity for correctly deciding, and an evaluating factor of thinking with complexity in steps of decision, or large quantity of factors of decision. Factors of the third skill category among the factors have an evaluating factor of performing with complexity or variety of methods or measures associated with the task, and an evaluating factor of cognition for difficulty in repetition of action for correct work.

Also, a risk diagnosing method of diagnosing risk of a task within a producing sequence is provided. In the risk diagnosing method, plural assessing criteria are predetermined including a product quality criterion, a cost loss criterion, a safety criterion, and an environmental criterion, for assessment of a predictable error of the task. The task is assessed with the plural assessing criteria, to obtain respectively influence ranks. An error influence level of the error is determined by processing the influence ranks in combination according to overall processing, influence of the error influence level being related to the producing sequence or a producing line having the producing sequence.

The product quality criterion is based on estimated influence to product quality upon occurrence of the error. The cost loss criterion is based on an estimated financial loss caused upon occurrence of the error. The safety criterion is based on estimated danger of the task in the error. The environmental criterion is based on estimated influence to environment upon occurrence of the error.

Furthermore, a characteristic evaluating method of evaluating a characteristic of a task within a producing sequence is provided. In the characteristic evaluating method, a constituent of learning in relation to difficulty in learning the task is determined. A constituent of risk is determined, in relation to influence of a predictable error of the task to the producing sequence or a producing line having the producing sequence. The characteristic of the task is determined by processing the constituent of learning and the constituent of risk in combination according to overall processing.

The plurality of the task is classified into a first task related to a range equal to or greater than a range of the producing sequence, and a second task related to installed machinery in the producing sequence, and the second task is subjected to evaluating of the characteristic.

The constituent of learning is evaluated by combined evaluation of a difficulty level of difficulty of the task, frequency of occurrence of the task, and constraint of available time required for the task.

The difficulty level is determined by steps which include categorizing a skill required for human resource to perform the task according to cognitive science to determine at least first, second and third skill categories, the first skill category being related to cognition of outer field information or acquisition of cognitive information, the second skill category being related to thinking or decision, and the third skill category being related to behavior or action. The task is evaluated with the first, second and third skill categories, to obtain respectively difficulty ranks, the difficulty ranks being processed in combination according to overall processing for difficulty level determination.

Plural factors are provided in each of the first, second and third skill categories. Factors of the first skill category among the factors have an evaluating factor of knowledge with sufficient quality or quantity for correctly recognizing a status, and an evaluating factor of cognition with degree of necessity of sensory organs for correctly recognizing a status. Factors of the second skill category among the factors have an evaluating factor of knowledge with sufficient quality or quantity for correctly deciding, and an evaluating factor of thinking with complexity in steps of decision, or large quantity of factors of decision. Factors of the third skill category among the factors have an evaluating factor of performing with complexity or variety of methods or measures associated with the task, and an evaluating factor of cognition for difficulty in repetition of action for correct work.

The constituent of risk is corrected according to a coefficient of modeled error occurrence determined according to the difficulty level.

The product quality criterion is based on estimated influence to product quality upon occurrence of the error. The cost loss criterion is based on an estimated financial loss caused upon occurrence of the error. The safety criterion is based on estimated danger of the task in the error. The environmental criterion is based on estimated influence to environment upon occurrence of the error.

Also, a task allocating method of allocating a task within a producing sequence is provided. In the task allocating method, a constituent of learning in relation to difficulty in learning the task is determined. A constituent of risk is determined in relation to influence of a predictable error of the task to the producing sequence or a producing line having the producing sequence. A characteristic of the task is determined by processing the constituent of learning and the constituent of risk in combination according to overall processing. The task is allocated to human resource in consideration of the characteristic of the task.

The human resource includes persons between which a degree of responsibility or duty is different.

Furthermore, a task improving method of improving a task within a producing sequence is provided. In the task improving method, a constituent of learning in relation to difficulty in learning the task is determined. A constituent of risk is determined in relation to influence of a predictable error of the task to the producing sequence or a producing line having the producing sequence. A characteristic of the task is determined by processing the constituent of learning and the constituent of risk in combination according to overall processing. The task is improved in consideration of the characteristic of the task.

Also, a task allocating method of allocating a task within a producing sequence is provided. In the task allocating method, plural assessing criteria are predetermined, including a product quality criterion, a cost loss criterion, a safety criterion, and an environmental criterion, for assessment of a predictable error of the task. The task is assessed with the plural assessing criteria, to obtain respectively influence ranks. An error influence level of the error is determined by processing the influence ranks in combination according to overall processing, influence of the error influence level being related to the producing sequence or a producing line having the producing sequence. The task is allocated to human resource in consideration of the characteristic of the task.

Furthermore, a task allocating computer-executable program for allocating a task within a producing sequence is provided. The task allocating computer-executable program includes code for determining a constituent of learning in relation to difficulty in learning the task. There is code for determining a constituent of risk in relation to influence of a predictable error of the task to the producing sequence or a producing line having the producing sequence. There is code for determining a characteristic of the task by processing the constituent of learning and the constituent of risk in combination according to overall processing. There is code for allocating the task to human resource in consideration of the characteristic of the task.

Also, a task allocating user interface for allocating a task within a producing sequence is provided. The task allocating user interface includes a region for determining a constituent of learning in relation to difficulty in learning the task. A region is for determining a constituent of risk in relation to influence of a predictable error of the task to the producing sequence or a producing line having the producing sequence. A region is for determining a characteristic of the task by processing the constituent of learning and the constituent of risk in combination according to overall processing. A region is for allocating the task to human resource in consideration of the characteristic of the task.

### BRIEF DESCRIPTION OF THE DRAWINGS

The above objects and advantages of the present invention will become more apparent from the following detailed description when read in connection with the accompanying drawings, in which:

FIG. 1 is a flow chart illustrating evaluation of characteristics of tasks;

FIG. 2 is a table illustrating hierarch of the tasks;

FIG. 3 is a chart illustrating correlated aspect of evaluating characteristics of the tasks;

FIG. 4 is a graph illustrating learning curves for the tasks;

FIG. 5 is a flow chart illustrating modeling of human cognitive processing of information;

FIG. 6 is a chart illustrating criteria of influence of errors;

FIG. 7 is a flow chart illustrating allocation of a task;

FIG. 8 is a flow chart illustrating improvement of the task;

FIGS. 9A and 9B are charts illustrating flow of steps for reducing a term of learning;

FIG. 10 is a chart illustrating correlated aspect of effects of evaluating characteristics;

FIG. 11 is a perspective view illustrating a lens fitted photo film unit produced in a production of the invention; and

FIG. 12 is an exploded perspective view illustrating the lens fitted photo film unit.

### DETAILED DESCRIPTION OF THE PREFERRED EMBODIMENT(S) OF THE PRESENT INVENTION

A preferred embodiment is described. Two types of workers exist, and include regular employees of a manufacturer, and leased workers who originally belong to an exterior company or agency of human resource, and have been sent to the manufacturer. In a producing line, workers of the two types work in mixture, to perform tasks. In the invention, characteristics of tasks are evaluated. Note that the term of task is used to stand for one of smaller portions included in one of producing sequences which are interconnected to constitute the producing line.

In Table 1, examples of tasks in photo film producing line are indicated. The line may be a producing line for producing any product or material. The tasks of the photo film producing line are grouped into tasks of working, tasks of transport and installation, and tasks of inspection. The tasks of working are such for producing products actually. The tasks of transport and installation are to transport or administer products and also various devices and materials for production. The tasks of inspection are to inspect completed products and failing products and to manage those. Also, grouping in a second manner defines a group of a regular tasks and temporary tasks. The regular tasks are performed in a normal state of production. The temporary tasks are performed at the time of changing over product types, and managing errors or maintaining the line.

TABLE 1
EXAMPLES OF TASKS IN PHOTO FILM PRODUCING LINE

| | REGULAR TASKS | TEMPORARY TASKS |
|---|---|---|
| WORKING | Changes of types, supply | Recovery of breakage of |
| | of parts, & periodical | machinery (darkroom), & |
| | inspection | test of changing factors |
| TRANSPORT & | Management of products | Transport of part units |
| INSTALLATION | & parts for quantity & | for maintenance, & |
| | quality, & transport of | transport of measuring |
| | parts & samples | device |
| INSPECTION | Inspection of | Primary decision upon |
| | functioning of photo | occurrence of errors, & |
| | film, & inspection of | management of producing |
| | size, weight & density | lot of occurrence of |
| | | errors |

The following is features of the photo film producing line analyzed according the three viewpoints of A, Environment of working, B, Installation and producing system, and C, Operation.

[A, Environment of Working]

1. Environment of the darkroom for numerous processes adapted to photosensitive material

2. Complicated system of conduits and layout of installation as a result of successive addition

[B, Installation and Producing System]

1. Conditioned control of machinery of high precision

2. Test production in a plant of mass production

3. Quality control and quantity control of raw materials of numerous types

4. System in a complicated structure with a considerable length

[C, Operation]

1. Changes in factors according to changes of raw materials or products and improvement of productivity

2. Assurance of quality of functioning

3. Operation of decision and combined operation by considering a flow and condition in an administration room

As described above, a material producing line, such as photo film producing line, has distinct features from those of generally used producing lines of other products because of highly specialized property, complexity and possibility of frequent modifications. As products of numerous kinds are produced from materials obtained from the line, maturity of production of the line has been desired in public as fundamental industry. Also, the environment of production is subjected to great changes in designs of products for improvement, transfer of the products, and changes in quantity because of shortness in commercial circulation, which is in addition to the increase in the number of retiring workers, and the increase in the number of leased workers different from regular employees. In view of those, innovation of the factory and low-cost operation are desired. Various attempts have been made for low-cost operation. For example, a producing sequence of an autonomous type, and multi process sequence are suggested. The system of fixed number of workers or system of division of tasks is reconsidered. Rotation of workers between the sequences or between factories, and utilization of leased workers are considered. Consequently, combinations between difficulty levels of tasks with workers should be optimized.

TABLE 2
METHODS OF EVALUATION OF WORK IN TASKS

| METHODS OF | CHARACTERISTICS OF | CRITERIA OF |
|---|---|---|
| EVALUATION | METHODS | CONVERSION |
| EVALUATION OF | EVALUATION OF SHAPES | QUANTIFICATION OF |
| ASSEMBLING PROPERTY | & SEQUENCES OF PARTS, | ASSEMBLING PROPERTY |
| | MICROSCOPIC | IN POINTS |
| EVALUATION OF | EVALUATION OF | PHYSICAL LOAD & |
| QUANTITY & LOAD OF | INTENSITY OF WORK & | METABOLISM OF ENERGY |
| WORK IN TASKS | WORKING TIME IN | |
| | TASKS, BOTH | |
| | MACROSCOPIC & | |
| | MICROSCOPIC | |
| EVALUATION OF | EVALUATION OF | QUANTIFICATION OF |
| QUALITY OF WORK IN | DIFFICULTY OF WORK IN | DIFFICULTY IN POINTS |
| TASKS | TASKS, MACROSCOPIC | |

In order to optimize combination of difficulty levels and workers, suitable evaluation of tasks is essentially required. In Table 2, examples of methods of evaluating tasks include the evaluation of assembling property (1), the evaluation of quantity and load of work in tasks (2), and the evaluation of quality of work in tasks (3). The evaluation of assembling property (1) is unsuitable for the purpose because its important factors are shapes of parts and products, sequences of assembly and suitability of a product itself for production. The evaluation of quantity and load of work in tasks (2) is unsuitable in view of an automated producing line despite its relevancy to intensity of work and evaluation of time. Therefore, in taking those into consideration, the evaluation of quality of work in tasks (3) is focused in the invention in which characteristics of tasks are evaluated according to difficulty levels of tasks.

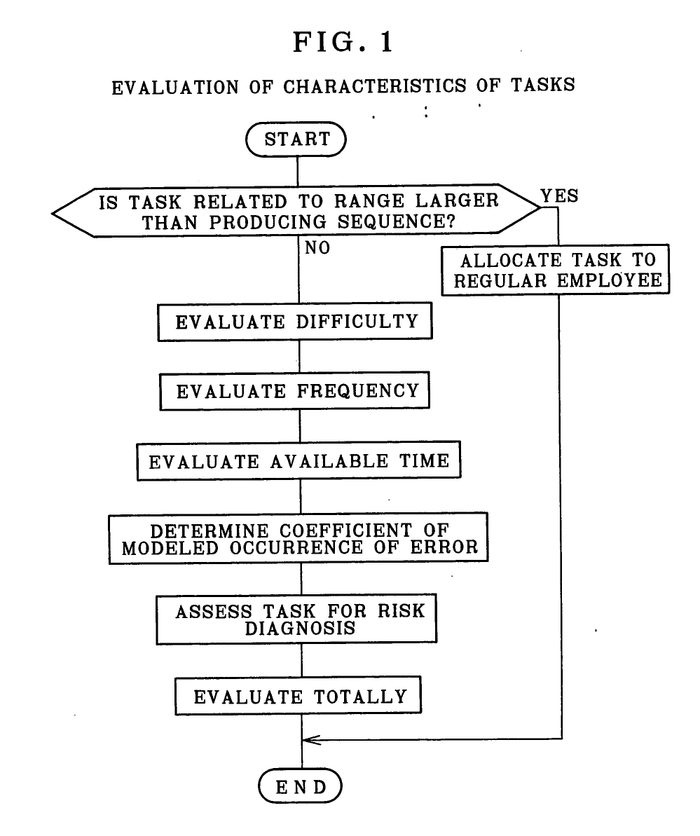

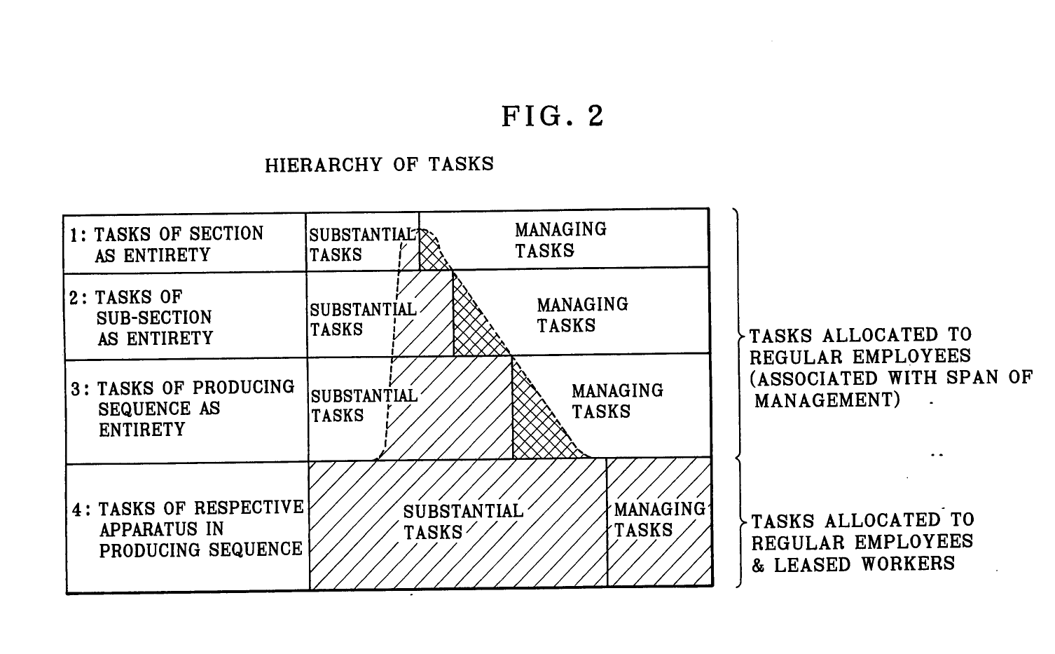

In FIG. 1, a flow of evaluation of characteristics of tasks is illustrated. At first, it is judged whether one task should be evaluated for its characteristics. This is because one producing line has a complicated structure where a target task should be specified for location, the producing line including plural sections, each of which includes plural sub-sections, each of the sub-sections including plural producing sequences. As illustrated in FIG. 2, tasks included in operation of the producing line are organized in four phases, namely tasks of a section as entirety, tasks of a sub-section as entirety, tasks of a producing sequence as entirety, and tasks of a respective apparatus in each one producing sequence. Also, the tasks are grouped in substantial tasks and managing tasks. The substantial tasks are related directly to production of products. The managing tasks are to manage the substantial tasks.

In the known producing line, tasks are allocated to employees and leased workers between the hatched regions and the cross-hatched regions. However, tasks of the cross-hatched regions are tasks relevant to a range over the entirety of producing sequences, and are unsuitable for allocation to leased workers, because of strict requirement of responsibility of final decision, instruction and verification (1), negotiation with other divisions of the manufacturer (2), and security of confidentiality (3). In the invention, tasks relevant to a range over the entirety of producing sequence (hereinafter referred to tasks of the span of management) are predetermined. Tasks of the span of management are kept allocated to employees. Remaining tasks not associated with the span of management are allocated to employees and leased workers suitably. Unnecessary portions of the evaluation of characteristics can be eliminated, as work for the span of management can be removed in the evaluation of characteristics.

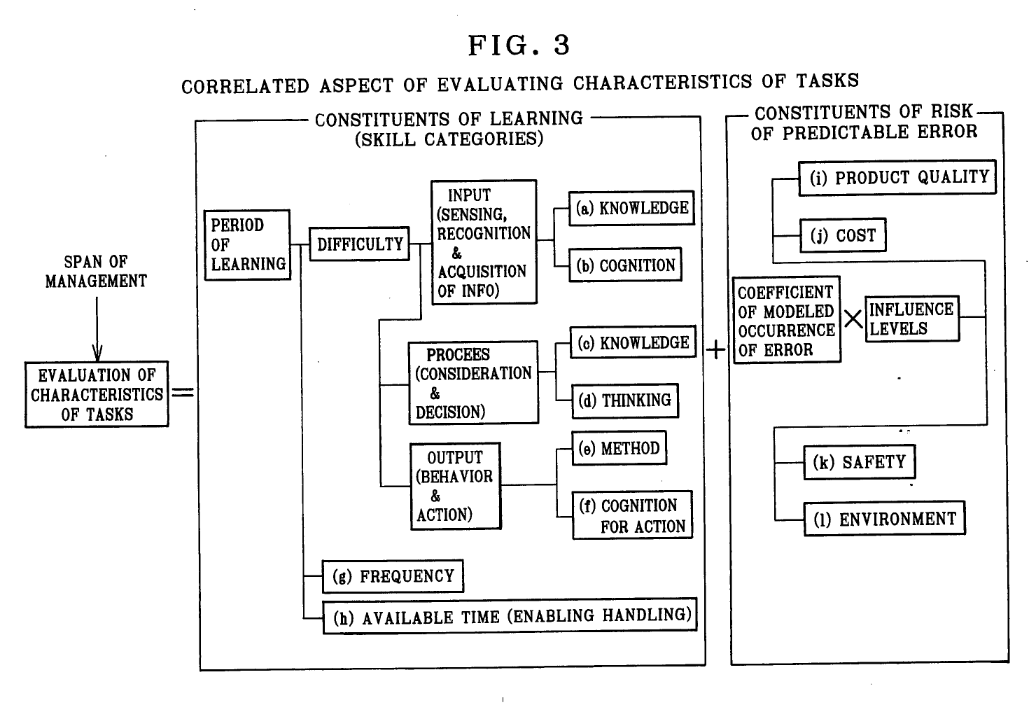

In FIG. 3, correlation of factors in evaluating characteristics of tasks is illustrated. A problem of leased workers is a limited period of working as staff, so they must be trained quickly by quick learning so as to work with high productivity early. Thus, the skill categories of learning are used for evaluating characteristics of tasks. Influence of predictable errors created in each of tasks is also a standard for representing importance of the task. Thus, constituents of risk of errors are used for evaluating characteristics of tasks.

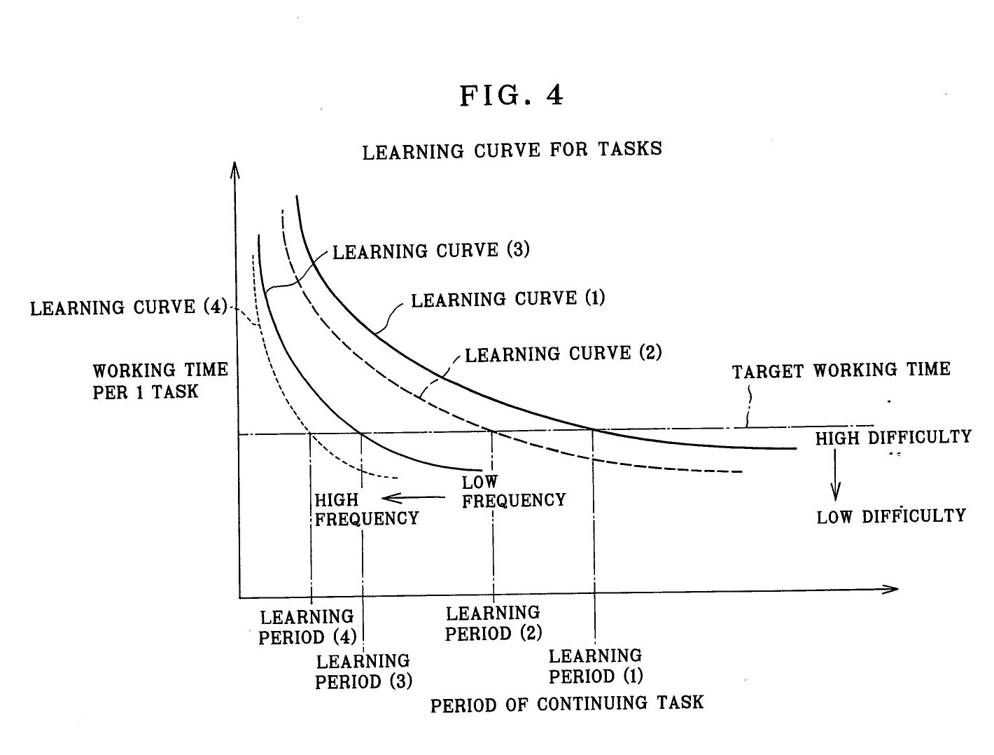

To extract factors of evaluation of the factors of learning, learning curves are used as a method of expressing a state of learning. In FIG. 4, learning curves are defined on the basis of difficulty level, frequency and working time. The difficulty of a task is derived from difficulty of property of the task. A learning period of the tasks changes according to a level of the difficulty. Frequency is the number of times of repeating the task per unit time, and independent from the difficulty. A learning period of the tasks changes according to the frequency. Let difficulty of a first task be high. A worker may perform the first task for many times during a short term, and then will learn the skill of the first task comparatively rapidly. In contrast, skill of even an easy task may be forgotten if frequency of occurrence of the task is as small as one time per year. The target working time is working time required for performing the task. In case of an equal difficulty, a difference occurs in a learning period between a first situation where short term learning is desired in view of great influence to other tasks, and a second situation which is free from constraint of time.

The horizontal axis is taken for the total period of continuing a task. The vertical axis is taken for the working time per one task. According to the learning curve (1), the degree of learning is deepened with time of continuation. The working time per one time gradually decreases. A value of target working time is determined, and considered with the learning curve (1). Thus, a learning period (1) is obtained by the consideration as period in which handling of the task within target working time has been learned. A learning curve (2) relates to a task with a lower difficulty than a difficulty of the task of the learning curve (1). A learning period (2) is obtained from the learning curve (2) as period in which handling of the task within the target working time of the learning period (1) has been learned. The learning period (2) is found shorter than the learning period (1).

A learning curve (3) relates to a task with low frequency of occurrence. A learning curve (4) relates to the same task as the learning curve (3) but with high frequency. It is observed with those curves that the learning period changes according to changes in the frequency.

The difficulty level of working according to the invention is conceived by the steps described below on the basis of learning curves. The difficulty level of which evaluation is specifically difficult is fundamentally based on the modeling of human cognitive processing of information.

1. Evaluation of the difficulty level of working as property of a task itself

2. The difficulty level of working is defined mainly as an index of ease of the learning.

Period of leaning is defined as (difficulty level)×(working frequency)×(working time per task).

3. Modeling of a task according to INPUT-PROCESS-OUTPUT of the modeling of human cognitive processing of information in cognitive science

4. Rating modeled task with ranks of the difficulty level by use of scores

5. Systematization of evaluating standards between producing sequences

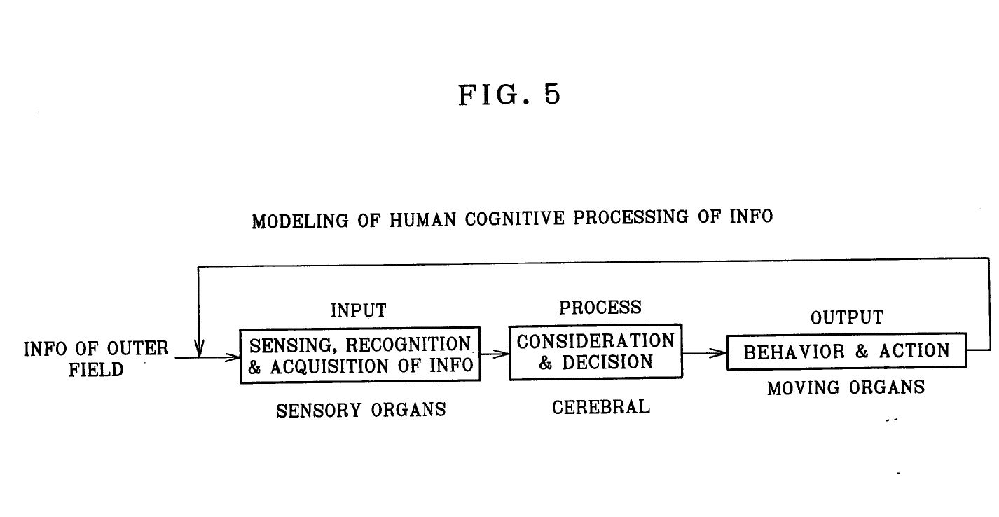

In FIG. 5, modeling of human cognitive processing of information is illustrated on the basis of cognitive science. A human being has sensory organs to perceive information of the exterior, and to acquire cognitive information, and cerebral to think and decide, and moving organs to move. According to those, function of a human being is grouped into three skill categories which are an INPUT skill category, PROCESS skill category, and an OUTPUT skill category.

TABLE 3
CONSTITUENTS OF LEARNING (SKILL CATEGORIES)

| | INPUT: | | |
|---|---|---|---|
| | SENSING, | | |
| | RECOGNITION & | PROCESS: | OUTPUT: |
| | ACQUISITION OF | CONSIDERATION | BEHAVIOR & |
| CONDITIONS | INFO | & DECISION | ACTION |
| A: PERSONAL | PHYSICAL | MEMORIZATION | MEMORIZATION |
| RESOURCE & | SENSING | JUDGEMENT | PHYSICAL SKILL |
| CONDITION OF | MEMORIZATION | PERSONALITY | OF MUSCLES, |
| WORKER | PERSONALITY | MOTIVATION | QUICKNESS & |
| (PERSONAL | MOTIVATION | RESPONSIBILITY | STAMINA |
| RESOURCE & | RESPONSIBILITY | | SKILL OF |
| CONDITION | | | COGNITION |
| WHICH WORKER IS | | | SKILL OF |
| REQUIRED TO | | | COMMUNICATION |
| HAVE PRIOR TO | | | PERSONALITY |
| LEARNING TASK) | | | MOTIVATION |
| | | | RESPONSIBILITY |
| B: | KNOWLEDGE | KNOWLEDGE | METHOD |
| CONSTITUENTS | TRAINED | THINKING | TRAINED |
| OF DIFFICULTY | COGNITION | | COGNITION |
| OF WORK | | | |
| (CONDITION | | | |
| WHICH WORKER IS | | | |
| REQUIRED TO | | | |
| HAVE DURING | | | |
| LEARNING OF | | | |
| TASK, & | | | |
| ACHIEVING | | | |
| LEVEL) | | | |

In Table 3, results of factors of evaluation of difficulty levels of a task are indicated according to the modeling of human cognitive processing of information described above. Difficulty in the respective processes is a factor influencing learning. Factors supposed as constituents in the processes are picked up. It is noted that the difficulty level for use in the invention does not evaluate the personal possibility of each worker, but is used for setting a condition and target level required for performing tasks related to machinery on the basis of difficulty solely derived from property of the task itself. In the present embodiment, factors on the second uppermost line A in the table such as memorization, personality and motivation are excluded as personal characteristics, because of lack of influence to learning time derived from difficulty levels of tasks. In contrast, factors on the lowest line B in the table are derived essentially in the invention, such as knowledge, cognition, thinking and methods.

TABLE 4
Definitions of Factors of Evaluation of Constitu-
ents of Learning (Skill Categories)

| | | ASPECT OF EVALUATION | | | |
|---|---|---|---|---|---|
| FACTORS OF EVALUATION | | S1 | S2 | S3 | S4 |
| INPUT: | (a) | A1 | A2 | A3 | A4 |
| COGNITION, | KNOWLEDGE | | | | |
| RECOGNITION & | (b) | B1 | B2 | B3 | B4 |
| ACQUISITION | COGNITION | | | | |
| OF INFO | | | | | |
| PROCESS: | (c) | C1 | C2 | C3 | C4 |
| CONSIDERATION | KNOWLEDGE | | | | |
| & DECISION | (d) | D1 | D2 | D3 | D4 |
| | THINKING | | | | |
| OUTPUT: | (e) | E1 | E2 | E3 | E4 |
| BEHAVIOR & | METHOD | | | | |
| ACTION | (f) | F1 | F2 | F3 | F4 |
| | COGNITION | | | | |
| | FOR | | | | |
| | ACTION | | | | |

| | | | | |
|---|---|---|---|---|
| (g) FREQUENCY | G1 | G2 | G3 | G4 |
| (h) AVAILABLE TIME | H1 | H2 | H3 | H4 |
| (ENABLING HANDLING) | | | | |

In the table, reference signs represent the following descriptions.

S1: Senses of factors of evaluation and definition of regions

S2: Viewpoints and measures in evaluation

S3: Improvement at the time of high score in evaluation

S4: Examples of measures

A1: Knowledge with sufficient quality and quantity for correctly recognizing status (normal or abnormal status for inspection)

A2: Range, depth and precision of knowledge, and difficulty in expressing knowledge

A3: To reduce required knowledge, remove necessity of memorizing required knowledge, and facilitate learning of knowledge

A4: Formulation and systematization, quick manual and check sheet, and facilitated system for search, and learning video

B1: Degree of necessity of sensory organs required for correctly recognizing status (quality and quantity of knowledge)

B2: Levels of requirement of sensory organs, and difficulty in repetition of cognition in sensory organs (desirable precision)

B3: To remove necessity of recognizing status, facilitate recognition of status, and facilitate learning of cognition

B4: Automation for cognition with sensory organs, enlarged display and quantified display with threshold, and simulated experience (for training)

C1: Knowledge with sufficient quality and quantity for correctly deciding (with examples of decision and past findings)

C2: Range, depth and precision of knowledge, and difficulty in expressing knowledge

C3: To reduce required knowledge, remove necessity of memorizing required knowledge, and facilitate learning of knowledge

C4: Formulation and systematization, quick manual and check sheet, and facilitated system for search, and learning video

D1: Complexity in steps of decision, and large quantity of factors of decision

D2: Complexity in deciding pattern, and difficulty in expressing deciding process

D3: To remove necessity of decision, facilitate decision, and facilitate learning of decision

D4: Automation of decision, clarification of thinking process, quantification and simplification of index and categories of criteria, and simulated experience (with FAQ)

E1: Complexity and variety of methods and measures associated with task (quality and quantity of knowledge)

E2: Complexity of sequences and their No. of steps (quantity of their knowledge), required precision, and difficulty in expressing methods and measures

E3: To reduce required knowledge, remove necessity of memorizing required knowledge, and facilitate learning of knowledge

E4: Formulation and systematization, quick manual and check sheet, and facilitated system for search, and learning video

F1: Difficulty in repetition of action for correct work

F2: Requirement of training, difficulty in physical action, and levels of requirement of physical action

F3: To remove necessity of cognition for behavior, facilitate repetition of behavior, and facilitate learning of cognition for behavior

F4: Simplification of action, reduction of action to one touch, removal of need of precision in positioning, and simulated experience (for repeating action)

G1: Frequency of scheduled tasks and frequency of chances of performing tasks

G2: Frequency of chances of performing tasks, and ease in keeping of attained learning level

G3: To increase scheduled experience, and increase parties having the same skill category

G4: Off line teaching, simulated experience and specialized operator in certain field

H1: Constraint in time of task and limited timing

H2: Constraint in available time of task, and degree of freedom in timing

H3: To reduce work of task with limited time or timing

H4: Share with outer staff and off line task

In Table 4, definitions of factors of evaluation of constituents of learning (skill categories) are indicated, together with aspect of evaluation and viewpoints and measures in evaluation. In Table 5, aspect of evaluation of difficulty, and its viewpoints are illustrated. In the INPUT skill category, the factor of (a) knowledge is knowledge with sufficient quality and quantity for correctly recognizing a status. The factor of (b) cognition is degree of necessity of sensory organs required for correctly recognizing a status. In the PROCESS skill category, the factor of (c) knowledge is knowledge with sufficient quality and quantity for correctly deciding. The factor of (d) thinking is complexity in steps of decision, and large quantity of factors of decision. In the OUTPUT skill category, the factor of (e) method is complexity and variety of methods and measures associated with the task. The factor of (f) cognition for action is difficulty in repetition of action for the correct work. The factor of (g) frequency is frequency of scheduled tasks and frequency of chances of performing. The factor of (h) available time (enabling handling) is constraint in time of the task and limited timing. Therefore, evaluation of difficulty can be objective and correct owing to the use of the .above defined factors.

TABLE 5
ASPECT OF EVALUATION OF DIFFICULTY

| FACTORS OF | | | |
|---|---|---|---|
| EVALUATION | | VIEWPOINT | DESCRIPTION |
| INPUT | KNOWLEDGE | RANGE | Evaluation as to extending |
| | | | only in a limited range, or the entirety of |
| | | | a specific producing sequence, or |
| | | | plural producing sequences including |
| | | | others. |
| | | DEPTH | Evaluation as to relation to |
| | | | knowledge of terminology & |
| | | | aspects of phenomena, or |
| | | | further to knowledge of |
| | | | structural or designing |
| | | | background of products & |
| | | | machinery. |
| | | PRECISION | Evaluation as to allowance of |
| | | | rough understanding or strict |
| | | | requirement of deep |
| | | | knowledge. |
| | | OTHERS | The knowledge is limited to |
| | | | such required for detection. |
| | | | How the obtained info should |
| | | | be read is regarded as |
| | | | knowledge in the PROCESS skill |
| | | | category. |
| | COGNITION | NECESSITY | Rating of 0 point in case an |
| | | OF TRAINING | unskilled person does not give |
| | | | a specific difference |
| | | | according to criterion of a |
| | | | level of cognition for daily |
| | | | life. Rating of 1-5 points in |
| | | | case of necessity of |
| | | | specialized training for |
| | | | cognition in the specific |
| | | | producing sequence in |
| | | | patterned or unpatterned |
| | | | cognition. |
| | | SPECIFIC | Evaluation as to whether a |
| | | DIFFERENCE | specific difference is likely |
| | | | to occur, & whether only |
| | | | limited staff can handle. |
| | | PRECISION & | Evaluation of requirement of |
| | | DIFFICULTY | high precision. |
| PROCESS | KNOWLEDGE | RANGE | Evaluation as to extending |
| | | | only in a limited range, or the |
| | | | entirety of a specific |
| | | | producing sequence, or plural |
| | | | producing sequences including |
| | | | others. |
| | | DEPTH | Evaluation as to relation to |
| | | | knowledge of terminology & |
| | | | aspects of phenomena, or |
| | | | further to knowledge of |
| | | | strctural or designing |
| | | | background of products & |
| | | | machinery. Evaluation as to |
| | | | whether one should be |
| | | | acquainted with phenomena, |
| | | | causes & measures upon |
| | | | occurrence of errors. |
| | | PRECISION | Evaluation as to allowance of |
| | | | rough understanding or strict |
| | | | requirement of deep |
| | | | knowledge. |
| | | OTHERS | Knowledge is evaluated if |
| | | | required for deciding |
| | | | succeeding behavior. |
| | THINKING | COMPLEXITY | Evaluation of Nos. of inputs |
| | | | & outputs such as 1:1 or |
| | | | plurality vs. plurality, as |
| | | | well as complexity of their |
| | | | relationships. Evaluation |
| | | | of necessity of quick |
| | | | responsiveness. |
| | | PRECISION | Evaluation of any one of only |
| | | | allowing best decision, & |
| | | | allowing deciding results |
| | | | with tolerable widths, & |
| | | | deciding as it is irrespective |
| | | | of values. |
| | | EVALUATION | Possibility of evaluation |
| | | OF RESULTS | with reference to specific |
| | | (WITH | criteria as to whether a |
| | | PRECISION) | result of consideration is |
| | | | good or not. |
| OUTPUT | METHOD | NO. OF STEPS | Evaluation as to whether the |
| | | | steps are difficult to |
| | | | memorize due to complexity, or |
| | | | as to whether a result will be |
| | | | reached by normal trial. |
| | | FINE | Likeliness of differences |
| | | DIFFERENCES | occurring according to small |
| | | OR | difference in the method. |
| | | PRECISION | |
| | | SPECIALTY | Evaluation as to necessity of |
| | | | special qualification or |
| | | | experience, for example, |
| | | | driving a fork lift truck, |
| | | | manipulating a computer, |
| | | | maintaining or the like. |
| | COGNITION | NECESSITY | Rating of 0 point in case an |
| | | OF TRAINING | unskilled person does not give |
| | | | a specific difference |
| | | | according to criterion of a |
| | | | level of cognition for daily |
| | | | life. Rating of 1-5 points in |
| | | | case of necessity of |
| | | | specialized training for |
| | | | cognition in the specific |
| | | | producing sequence in |
| | | | patterned or unpatterned |
| | | | cognition. |
| | | SPECIFIC | Evaluation as to whether a |
| | | DIFFERENCE | specific difference is likely |
| | | | to occur, & whether only |
| | | | limited staff can handle. |
| | | PRECISION & | Evaluation of requirement of |
| | | DIFFICULTY | high precision. |
| | | OTHERS | Evaluation limited to |
| | | | cognition required for |
| | | | behavior. |

In Table 6, ranks of difficulty level, frequency (g) and available time (h) are indicated. The difficulty level of working is ranked in six ranks, which are promptly learnable (0 point), easy to learn (1 point), possible to learn (2 points), possible to learn with much work (3 points), learnable with possible difficulty (4 points), and difficult to learn (5 points). Furthermore, the frequency (g) is ranked in six ranks, which are one or more times per hour (0 point), at least plural times per one shift of shift work, and at most one time per hour (1 point), one time per one shift of shift work (2 points), at least one time per week, and at most one time per one shift of shift work (3 points), at least one time per month, and at most one time per week (4 points), and one time or less per month (5 points). Additionally, the available time (h) is ranked in six ranks, which are very long available time (0 point), long available time (1 point), moderately long available time (2 points), short available time (3 points), very short available time (4 points), and requirement of urgency or lack of available time (5 points) with priority over other tasks. Those factors are rated according to the fixed ranks of evaluation of the list, so as to evaluate the difficulty level objectively in a quantitative manner.

TABLE 6
FACTORS OF EVALUATION OF CONSTITUENTS OF LEARNING
(SKILL CATEGORIES)

| | | RANKS OF EVALUATION | | | | | |
|---|---|---|---|---|---|---|---|
| FACTORS OF EVALUATION | | R0 | R1 | R2 | R3 | R4 | R5 |
| INPUT: | (a) KNOWLEDGE | A0 | A1 | A2 | A3 | A4 | A5 |
| SENSING, | (b) COGNITION | B0 | B2 | B2 | B3 | B4 | B5 |
| RECOGNITION & | | | | | | | |
| ACQUISITION OF | | | | | | | |
| INFO | | | | | | | |
| PROCESS: | (c) KNOWLEDGE | C0 | C1 | C2 | C3 | C4 | C5 |
| CONSIDERATION | (d) THINKING | D0 | D1 | D2 | D3 | D4 | D5 |
| & DECISION | | | | | | | |
| OUTPUT: | (e) METHOD | E0 | E1 | E2 | E3 | E4 | E5 |
| BEHAVIOR & | (f) COGNITION | F0 | F1 | F2 | F3 | F4 | F5 |
| ACTION | FOR ACTION | | | | | | |

| | | | | | | |
|---|---|---|---|---|---|---|
| (g) FREQUENCY | G0 | G1 | G2 | G3 | G4 | G5 |
| (h) AVAILABLE TIME (ENABLING | H0 | H1 | H2 | H3 | H4 | H5 |
| HANDLING) | | | | | | |

In the table, reference signs represent the following descriptions.

R0: 0, Promptly learnable

R1: 1, Easy to learn

R2: 2, Possible to learn

R3: 3, Possible to learn with much work

R4: 4, Learnable with possible difficulty

R5: 5, Difficult to learn

A0: Level with required knowledge of only names of machinery and products.

A1: Level with knowledge for driving producing line at a minimum level. Level with basic knowledge of products. Level with required knowledge of patterned solution of problems upon occurrence.

A2: Between 1 and 3

A3: Level with overall knowledge of entirety of producing sequence and machinery. Level with overall knowledge of entirety of products.

A4: Between 3 and 5

A5: Level with required knowledge of relationships or construction of machinery and products. Level of familiarity with performance and designing of products. Level with required knowledge of stored acquisition of cognition for information with fine differences.

B0: No specialized cognition. No need of training cognition.

B1: Existence of recognition or detection.

B2: Task to recognize a usual or regular pattern

B3: Task to recognize a usual or regular pattern and irregular pattern in mixed state

B4: Unpatterned task requires contribution of cognition.

B5: Long term training is required for task as cognition must be essentially trained.

C0: Level with required knowledge of only names of machinery and products. No knowledge is required for errors.

C1: Level with knowledge for driving producing line at a minimum level. Level with required knowledge of patterned solution of problems upon occurrence.

C2: Between 1 and 3

C3: Level with overall knowledge of entirety of producing sequence and machinery. Level with overall knowledge of entirety of products.

C4: Between 3 and 5

C5: Level with required knowledge of relationships or construction of machinery and products. Level of familiarity with performance and designing of products. Level with required knowledge of methodology and theories specialized for solving problems.

D0: No causes of specific consideration. Level of consideration is usual. No prior experience of consideration is required.

D1: Tasks can be treated with patterned thinking with simplicity. Prompt understanding is possible with slight experience.

D2: Tasks can be treated with patterned and exact thinking, or treated with unpatterned but simple thinking.

D3: Correct thinking is required in treating tasks with mixture of patterned thinking and unpatterned thinking with required correct thinking.

D4: Unpatterned thinking is required, but tasks can be treated with combinations of known rules and logic. Correct thinking is required inclusive of estimation and deduction.

D5: Tasks include elements without rules and logic. Also, correct thinking is required inclusive of estimation and deduction.

E0: Task requires only level of regular pattern in a simple and usual form.

E1: Task requires only medium low precision, and requires task steps of action which are simple, or task steps of small number.

E2: Between 1 and 3

E3: Task requires precision of a middle level, and requires task steps of action which are slightly complicated, or task steps of medium large number.

E4: Between 3 and 5

E5: Task requires high precision, and requires task steps of action which are complicated, or task steps of very large number.

F0: No specialized cognition. No need of training cognition.

F1: Minimum specialized cognition over the usual cognition level in action. A person with even slight experience can learn the cognition in action.

F2: The cognition in action of patterned cognition is used as factors of a task are ensured or repeated.

F3: The cognition in action in mixture of the patterned cognition and unpatterned cognition is used, with necessity of reliability and repetition of the task.

F4: The task needs reliability and repetition caused by the cognition in action of the unpatterned cognition for the task.

F5: The cognition in action of the unpatterned cognition is required for many types, wide range and high precision.

G0: 1 or more times per hour for 1 person

G1: At least plural times per 1 shift of shift work of 1 person, and at most 1 time per hour for 1 person

G2: 1 time per 1 shift of shift work of 1 person

G3: At least 1 time per week for 1 person, and at most 1 time per 1 shift of shift work of 1 person

G4: At least 1 time per month for 1 person, and at most 1 time per week for 1 person

G5: 1 time or less per month for 1 person

H0: Available capacity is considerably large in view of standard working time. Little influence occurs to other tasks even in case of great delay in the task. No need of concerning working time.

H1: The available capacity is sufficient in view of performing the task, but influence to progress occurs if delay of performing the task remains to succeeding shift of shift work.

H2: The standard working time must be concerned in task but still with the available capacity. Delay in performing the task may cause influence, but can be overcome.

H3: Constraint of working time as standard time exists, but with the available capacity. Delay in the task inevitably causes influence and cannot be overcome easily if repeated.

H4: Constraint of working time as standard time is strict, and does not leave remaining available capacity. Even a small delay for timing causes influence and cannot be overcome easily.

H5: Quick response is essential with priority over other tasks. The completion of the task should be as early as possible. If delay occurs in the response, huge influence and damage will be caused.

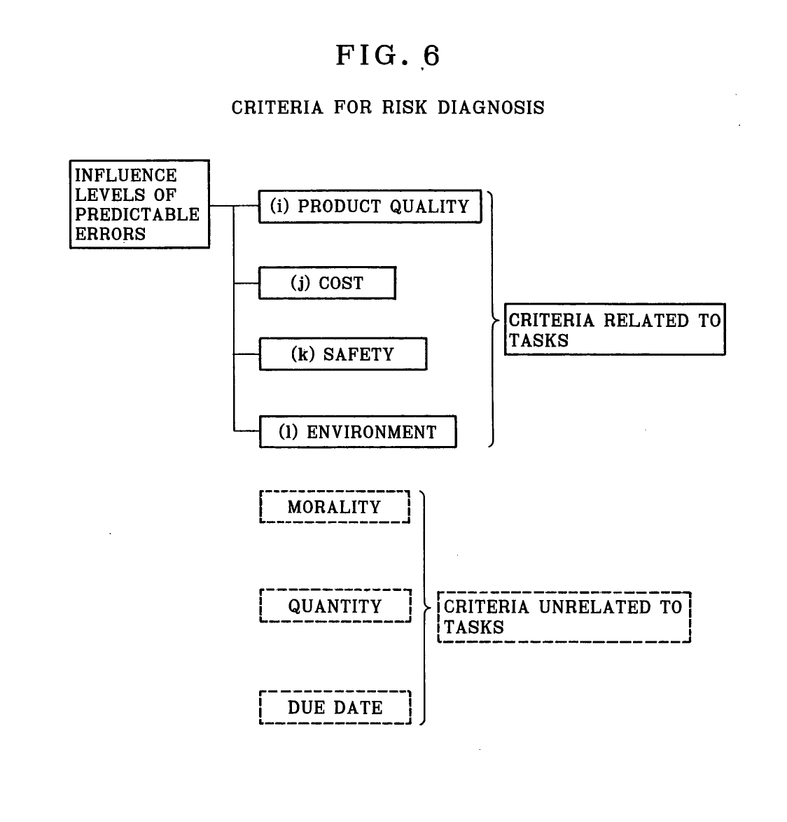

In FIG. 6, factors of being influenced by a predictable error in working of a task are illustrated. In the embodiment, four factors are criteria for assessing influence levels of an error, including (i) product quality, (j) cost, (k) safety, and (1) environment. In Table 7, definition, viewpoints and measures, improvement, examples of measures are indicated in relation to degree of influence of errors. The criterion of (i) PRODUCT QUALITY is a range of extension of influence upon occurrence of expected errors of task or errors found with unacceptability (NG). The criterion of (j) COST is a quantity of financial loss created upon occurrence of expected errors of task or errors found with unacceptability (NG). The criterion of (k) SAFETY is a degree of danger in task caused by expected errors of task. The criterion of (1) ENVIRONMENT is influence to environment caused by expected errors of task.

TABLE 7
DEFINITIONS OF CRITERIA FOR RISK DIAGNOSIS
OF PREDICTABLE ERRORS

| | | ASPECT OF | | | |
|---|---|---|---|---|---|
| CONSTITUENTS OF RISK IN | | ASSESSMENT | | | |
| ASSESSMENT | | S1 | S2 | S3 | S4 |
| RISK OF | (i) PRODUCT | I1 | I2 | I3 | I4 |
| INFLUENCE OF | QUALITY | | | | |
| ERRORS | (j) COST | J1 | J2 | J3 | J4 |
| | (k) SAFETY | K1 | K2 | K3 | K4 |
| | (l) | L1 | L2 | L3 | L4 |
| | ENVIRONMENT | | | | |
| DEGREE OF | COEFFICIENT OF | M1 | M2 | M3 | M4 |
| INFLUENCE OF | MODELED | | | | |
| OCCURRENCE | OCCURRENCE OF | | | | |
| | ERROR | | | | |

In the table, reference signs represent the following descriptions.

S1: Senses of factors of evaluation and definition of regions

S2: Viewpoints and measures in evaluation

S3: Improvement at the time of high score in evaluation

S4: Examples of measures

I1: Range of extension of influence upon occurrence of expected errors of task or errors found with unacceptability (NG)

I2: Range of influenced sections upon occurrence of errors

I3: To prevent occurrence of chain of errors, and prevent influence of quality from spreading

I4: Double check, prevention of inadvertent errors, bar code check, reduction of size of producing lot, ensured management with tracking No., and system for reliability within producing sequence

J1: Quantity of financial loss created upon occurrence of predictable errors of task or errors found with unacceptability (NG)

J2: Quantity of influenced products and quantity of loss upon occurrence of errors

J3: To prevent occurrence of chain of errors, and keep loss of the cost small even upon occurrence of any errors

J4: Double check, prevention of inadvertent errors, bar code check, reduction of size of producing lot, ensured management with tracking No., and system for reliability within producing sequence

K1: Degree of danger in task caused by predictable errors of task

K2: Degree of damage upon occurrence of injury

K3: To prevent occurrence of chain of errors, and keep influence small regarding safety even upon occurrence of any errors

K4: Fool proof safety, prevention of inadvertent errors, anti-dropping safety fence, safety cover, blade cover, protecting tool, and protecting eyeglasses or mask

L1: Influence to environment caused by predictable errors of task

L2: Range of influenced sections upon occurrence of failing operation

L3: To prevent occurrence of chain of errors, and keep influence small in environment even upon occurrence of any errors

L4: Fool proof maintenance of environment, prevention of inadvertent errors, interlock, warning, interior double check, and reconsideration of selection for outer abandonment

M1: Existence or lack of increase of errors upon changing over product types

M2: Existence or lack of additional consideration of influence with coefficient of 0-1, where 0 is a value for an unnecessary state of consideration, 0.5 is a value of wanting for a preferable consideration, and 1 is a value with must for a required consideration

M3: To prevent occurrence of errors even with unskilled workers at low level of task learning

M4: Reduction of the length of learning period, fool proof system, prevention of inadvertent errors, interlock, warning, and interior double check

In Table 8, ranks of assessment are indicated in association with criteria of assessing influence. In the embodiment, six (6) ranks are defined according to regions where influence o an error runs off, including a safe state without a problem (0 point), a region in a producing sequence (1 point), a region in a section (2 points), a region in a division (3 points), a region in a factory (4 points), and a region outside the company (5 points). As those ranks are rated according to the assessment criteria, it is possible to assess difficulty level quantitatively in an objective manner.

TABLE 8
CRITERIA FOR RISK DIAGNOSIS OF PREDICTABLE ERRORS

| | CONSTITUENTS OF RISK IN ASSESSMENT | | | |
|---|---|---|---|---|
| | (i) | | | |
| RANKS OF | PRODUCT | | | (l) |
| ASSESSMENT | QUALITY | (j) COST | (k) SAFETY | ENVIRONMENT |
| 0, NO | NO PROBLEM | NO | NO PROBLEM | NO PROBLEM |
| PROBLEM | | PROBLEM | | |
| 1, | LOW | Under | scratch | Slight fluid |
| PRODUCING | QUALITY IS | 100,000 | | flows off in |
| SEQUENCE | TREATABLE | yen | | producing |
| | WITHIN THE | | | sequence. |
| | PRODUCING | | | Slight fluid |
| | SEQUENCE. | | | is abandoned |
| | | | | as error. |
| 2, IN | LOW | 100,000-500,000 | incised wound | Slight fluid |
| SECTION | QUALITY | yen | or rash | flows off in |
| | RUNS OFF TO | | | section. |
| | NEXT | | | Much fluid is |
| | PRODUCING | | | abandoned as |
| | SEQUENCE. | | | error. |
| 3, IN | LOW | 500,000-2,000,000 | bleeding, | Much fluid |
| DIVISION | QUALITY | yen | inflammation | flows off in |
| | RUNS OFF TO | | or scald | section. |
| | ANOTHER | | | |
| | SECTION. | | | |
| 4, IN | LOW | 2-5 | bone fracture | Fluid flows |
| FACTORY | QUALITY | million | (squeezed or | off in the |
| | RUNS OFF TO | yen | struck by | factory. |
| | ANOTHER | | dropped | |
| | DIVISION. | | object) | |
| 5, OUTSIDE | LOW | Over 5 | amputation | Fluid flows |
| COMPANY | QUALITY | million | (task with | out of the |
| | RUNS OFF | yen | source of | factory. |
| | FROM THE | | motion or | |
| | COMPANY. | | blade) | |

To use predictable errors in evaluating characteristics of tasks, probability of occurrence of errors needs considering in addition of influence of errors. Table 9 is a list for indicating results of measuring probability of errors between regular employees and leased workers. Errors are classified into two types, inadvertent errors and serious errors. The inadvertent errors are errors committed in such an inadvertent manner or failure that one must have made something but has not actually made the same. The serious errors are errors other than inadvertent errors, and derived from shortage of required knowledge, or strict requirement of cognition or thinking at a range beyond a limit.

TABLE 9
RATIO OF ERRORS

| | TYPES OF ERRORS | | | |
|---|---|---|---|---|
| | INADVERTENT | | REMAINING ERRORS | |
| | ERRORS | | OR SERIOUS ERRORS | |
| | TYPES OF WORKERS | | | |
| | LEASED | EM- | LEASED | |
| | WORKERS | PLOYEES | WORKERS | EMPLOYEES |
| NO. OF | 3 | 13 | 6 | 5 |
| ERRORS | | | | |
| TOTAL OF | 90 | 518 | 90 | 518 |
| WORKERS | | | | |
| RATIO OF | 3.33% | 2.51% | 6.67% | 0.97% |
| ERRORS | | | | |

| | | |
|---|---|---|
| RATIO | 1:0.75 | 1:0.14 |
| BETWEEN | | |
| WORKER | | |
| TYPES | | |

There occur 16 inadvertent errors and 11 serious errors in the producing line of the embodiment. Among those, 13 inadvertent errors of employees are much more than 3 inadvertent errors of leased workers. However, a ratio of inadvertent errors of occurrence by the employees is near to a ratio of inadvertent errors of occurrence by the leased workers. A ratio of serious errors of occurrence by the leased workers is approximately seven (7) times as high as a ratio of serious errors of occurrence by the employees. It follows that the ratio of errors of leased workers is higher in relation to the specific tasks requiring knowledge, cognition and thinking.

In view of the probability of occurrence of predictable errors, influence of errors is compensated for according to difficulty levels of tasks. Among various tasks, a task with a high rank of difficulty can be regarded as a task which a leased worker finds it difficult to perform, and of which occurrence of error will create high influence. In Table 10, a value 1 is given as a coefficient of modeled occurrence of error when the score of one or more factors among the six is 4 or 5 points. A value 0 is given as a coefficient of modeled occurrence of error when the score of the six factors (a) to (f) is respectively 0-2 points, because little influence is estimated even if the error has occurred. A value 0.5 is given as a coefficient of modeled occurrence of error when the score of no factors among the six is 4 or 5 points, and score of one or more factors among the six is 3 points, because medium influence is estimated if the error has occurred. The score of each one of factors (a) to (f) of the influence levels is multiplied by the coefficient of modeled occurrence of error.

TABLE 10
COEFFICIENT OF MODELED OCCURRENCE OF ERROR

| | COEFFICIENT OF |
|---|---|
| | MODELED |
| SCORE OF EVALUATION OF | OCCURRENCE |
| DIFFICULTY LEVELS | OF ERROR |
| SCORE OF 1 OR MORE FACTORS AMONG | 1 |
| 6 IS 4 OR 5 POINTS | |
| SCORE OF NO FACTORS AMONG 6 IS | 0.5 |
| 4 OR 5 POINTS, & SCORE OF 1 OR | |
| MORE FACTORS AMONG 6 IS 3 POINTS | |
| SCORE OF 6 FACTORS IS 0-2 POINTS | 0 |

To evaluate characteristics of tasks, one suitable method of the evaluating methods should be selected for determination by combined consideration of skill categories of learning, and constituents of risk as criteria of influence levels of errors. As indicated in Table 11, there are plural candidate methods of the evaluation, which are (1) the total score evaluating method, (2) highest score evaluating method, (3) matrix evaluation, and (4) evaluation of multivariate analysis. Merits and demerits of those four are considered in view of suitability for the purpose of the invention. As a result, the total score evaluating method in which points of score of factors are added up is the most suitable of the four methods. Reasons of adopting the total score evaluating method are as follows. First, results of the total score evaluating method, when experimentally observed, are very likely to coincide with results of actual information personally acquired in the producing sequence. Secondly, differences in precision of the four evaluating methods are sufficiently small, the four having disadvantages distinct from one another. Thirdly, the total score evaluating method can give the most clarified results in the best manner in view of easy availability and quickness as a tool for practical use. Note that the total evaluating method is not limited to the total score evaluating method in the invention. It is possible to create a new composite method by combining two or more of the plural known methods, to utilize advantages of such methods.

TABLE 11

| TOTAL EVALUATING | | |
|---|---|---|
| METHOD | MERITS | DEMERITS |
| AS PER TOTAL SCORE | 1: Tasks can be | Even when a high rank |
| | ranked only by one | of one factor is |
| | simple criterion, so | outstanding, it is |
| | results in one | likely that the one |
| | dimension are easy | factor is overlooked |
| | to observe. 2: | among others. It is |
| | Tendencies are | likely that |
| | observable by | tendencies cannot be |
| | modeling with ranks | found easily even |
| | & given home | with the total score. |
| | position. | |
| AS PER HIGHEST | Even tasks with | Exceeding reaction |
| SCORE | points requiring | is somewhat likely to |
| | special care can be | occur. |
| | picked up | |
| | completely. | |
| MATRIX EVALUATION | 1: Improvement can | 1: Ranks of tasks are |
| (CONSTITUENTS OF | be easily planned. | not easily observed |
| LEARNING & RISK ARE | 2: Features & | due to two |
| TAKEN ON X & Y AXES, | tendencies of | dimensional view. |
| TO PLACE INFO OF | producing sequences | 2: Meanings of groups |
| TASK ON A | & sections can be | of ranking are not |
| COORDINATE SYSTEM | found observable by | determined. |
| OF TWO DIMENSIONS | two viewpoints. | |
| OF TWO FACTOR | | |
| CATEGORIES FOR | | |
| EVALUATION) | | |
| AS PER MULTIVARIATE | Effective in | Special analysis is |
| ANALYSIS (MTS, | preselection of | required, so |
| DISCRIMINANT | factors & effective | tendencies are |
| ANALYSIS & THE LIKE) | in reasoning that a | difficult to observe |
| | certain task should | even upon finding the |
| | be assigned to | score. Difficult to |
| | regular employees, | utilize for actual |
| | | use. |

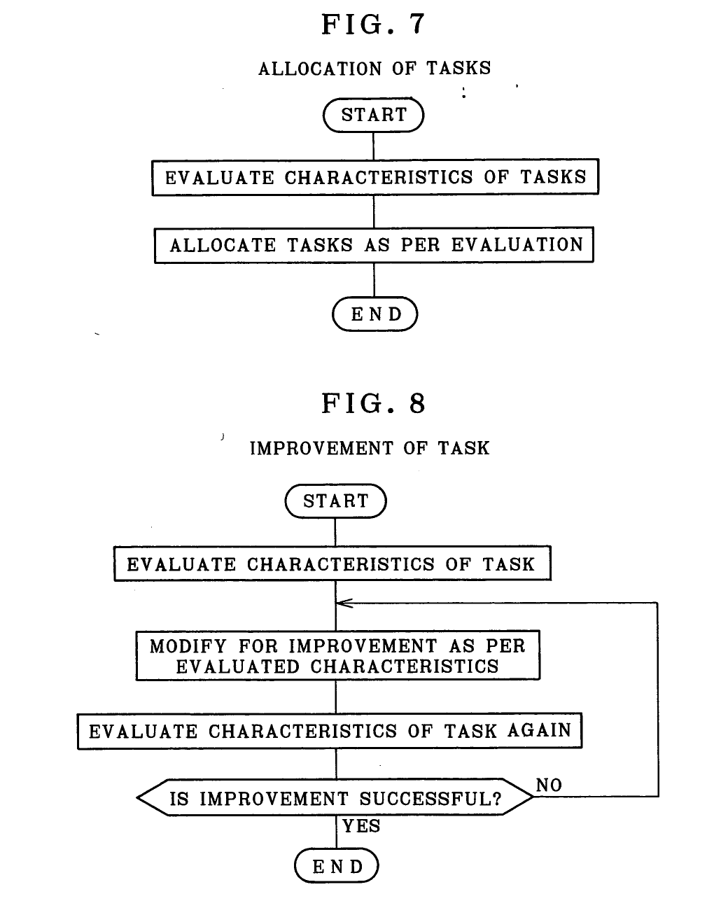

In FIG. 7, tasks after the total evaluation are allocated between employees and leased workers according to evaluated information. A problem arises in a selection among the levels of the tasks for allocation between employees and leased workers. According to the evaluation of characteristics of the tasks, the minimum of the score is 0 point because of the greatest ease and smallest influence of an error. The maximum of the score is 60 points because of the greatest difficulty and highest influence of an error. Thus, a task with the score of 30 points or more can be allocated to employees. A task with the score under 30 points can be allocated to leased workers. Furthermore, it is possible that a task with the score of 0-20 points is allocated to leased workers. A task with the score of 30-60 points can be allocated to employees. A task with the score of 20-30 points can be allocated suitably by decision by an administrator. In any of those ideas, tasks are allocated by the score of the evaluation of characteristics of those, so as to allocate tasks without unevenness between sub-sections or other small units of the factory.

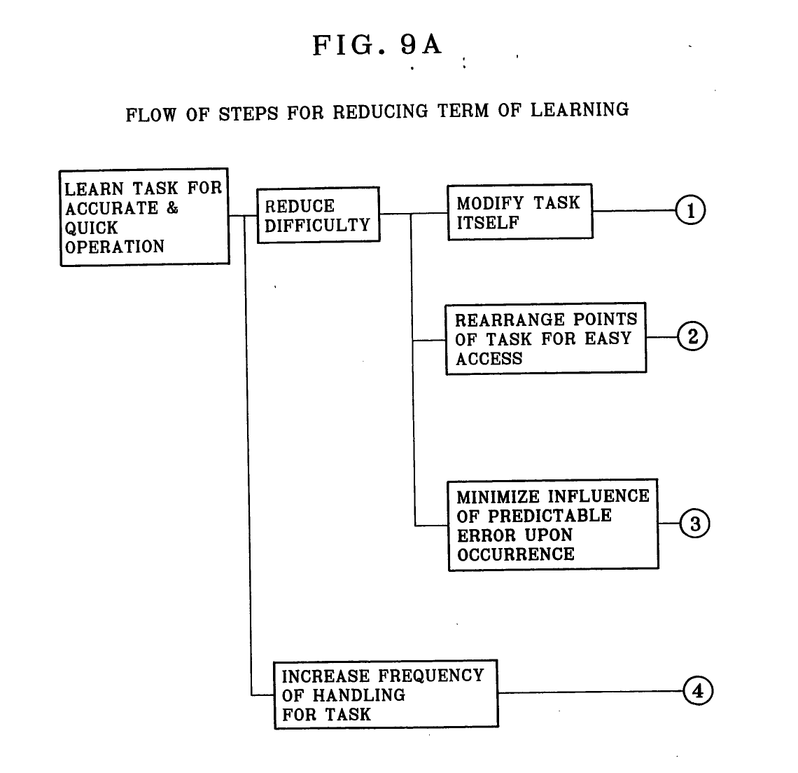

In FIG. 8, a flow of evaluation for the purpose of improvement of a task is illustrated. It is preferable to modify elements relevant to the task for improvement according to the evaluated characteristics, and then to evaluate characteristics of task again, so as to check a result of the improvement.

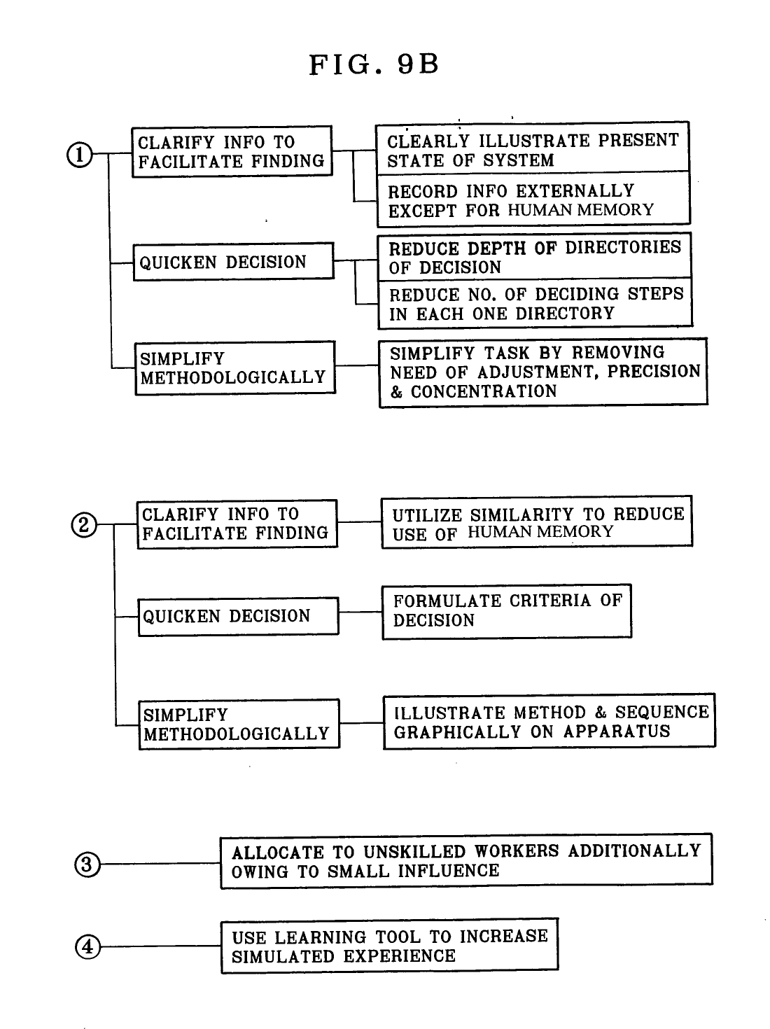

In FIG. 9, a flow of steps for reducing the term of learning is illustrated, together with specific ideas of modifying tasks or machinery. To reduce the term of learning, two methods are conceivable, which are to reduce difficulty, and to increase frequency of handling for a task. The methods of reducing difficulty include modifying of task itself, rearranging of points of task for easy access, and minimizing of influence of predictable error upon occurrence. Also, to minimize influence of an error upon occurrence is effective. The methods of increasing frequency of handling for a task include use of a learning tool to increase simulated experience. Consequently, improvement and modification according to the evaluation of features are suitable for requirement. Effects of the improvement can be verified objectively and quantitatively, so the improvement can be effective.

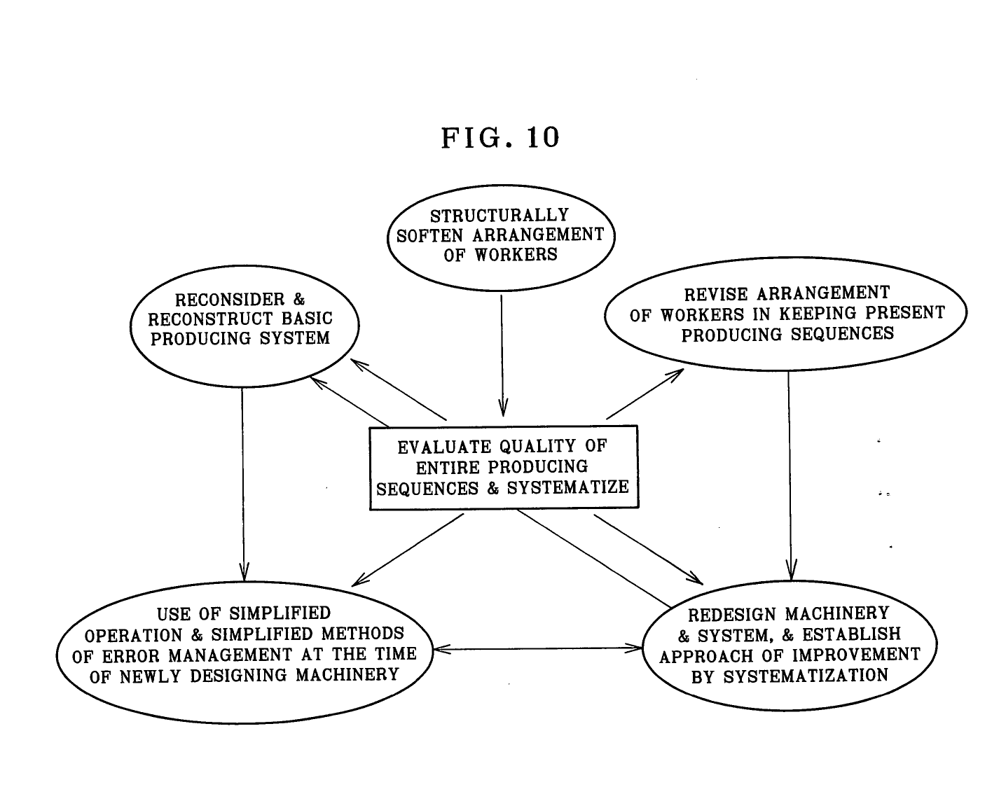

In FIG. 10, conception of effects of evaluating characteristics of the tasks is illustrated. One important purpose of the invention is structurally to soften arrangement of workers, so it is possible to revise the arrangement of workers in keeping the present producing sequences. Furthermore, the evaluation of the characteristics of the tasks and systematization of the tasks makes it possible to reconsider and reconstruct the basic producing system, to revise arrangement of workers in keeping the present producing sequences, and to use simplified operation and simplified methods of error management at the time of newly designing machinery, and to redesign machinery and system, and establish approach of improvement by systematization. Also, close correlation exists between those, to create further effects by synergism.

### EXAMPLES

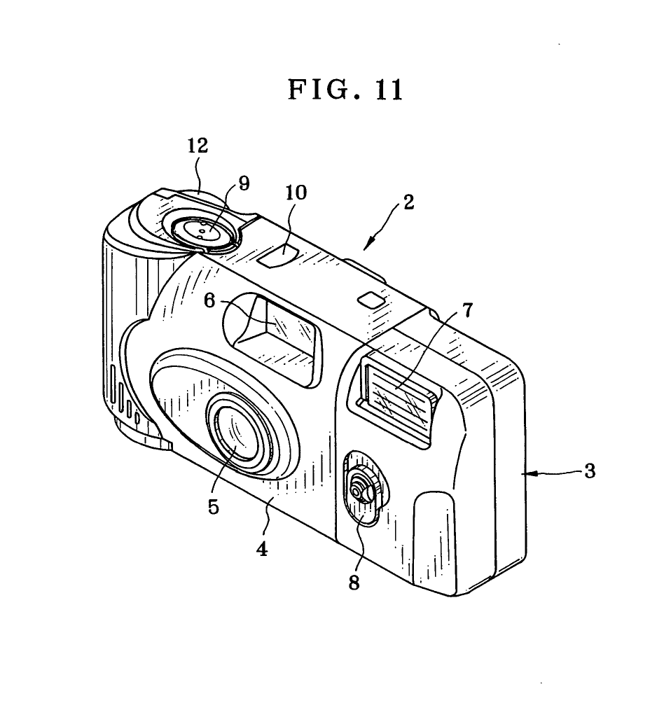

Examples of the evaluation of tasks are applied in a producing line for a lens fitted photo film unit 2. In FIG. 11, appearance of the lens fitted photo film unit 2 is illustrated. The lens fitted photo film unit 2 includes a housing 3 and a belt of sticker or label 4. The housing 3 is previously loaded with a photo film cassette in the course of manufacture. The sticker 4 is attached to the periphery of the housing 3. Openings are formed in the sticker 4 for uncovering portions of the housing 3.

In the front of the housing 3, there are a taking lens 5, a viewfinder 6, a flash light source 7, and a flash charger switch 8. On the upper face of the housing 3, there are a shutter release button 9 and a counter window 10. In the rear of the housing 3, a winder wheel 12 protrudes partially to the outside, for use in photo film winding of one frame after each exposure.

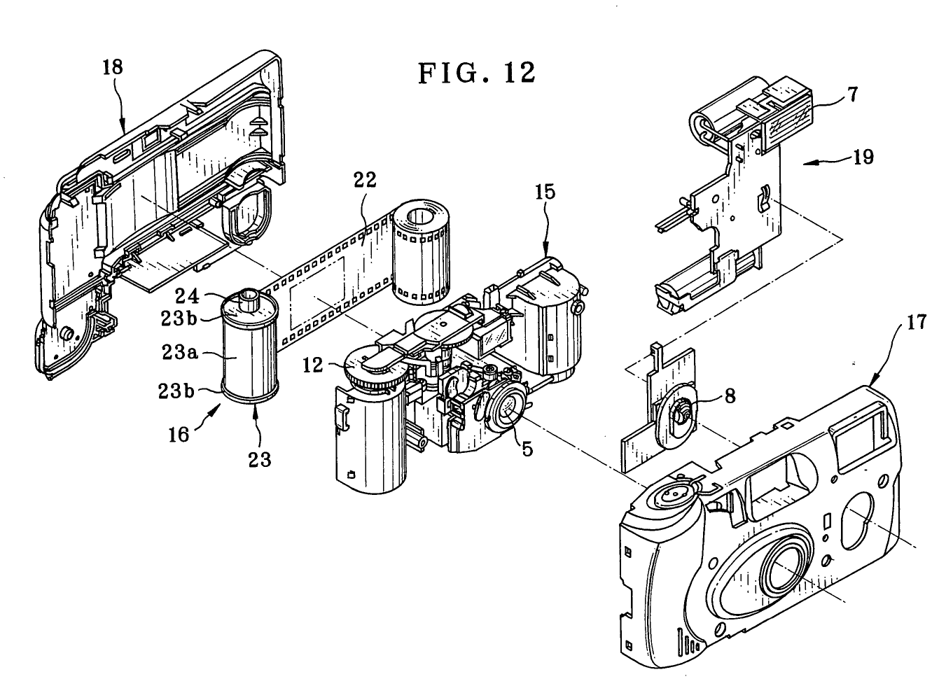

In FIG. 12, the lens fitted photo film unit 2 includes a main body 15, a photo film cassette or cartridge 16, a front cover 17, a rear cover 18 and a flash device 19. A first step of producing the lens fitted photo film unit 2 is to assemble a photographing mechanism on the main body 15. The flash device 19 is secured to the main body 15. The front cover 17 is fitted on the front of the main body 15. The photo film cassette 16 is inserted in the main body 15 through the rear thereof. The rear cover 18 is attached to the photo film cassette 16. The sticker 4 is attached finally, to obtain the lens fitted photo film unit 2.

The photo film cassette or cartridge 16 includes a strip of photo film 22 and a cassette shell 23 for containing the photo film 22. The cassette shell 23 includes a tubular shell body 23a, a pair of shell caps 23b and a spool 24. The shell caps 23b are fitted on end openings of the tubular shell body 23a. The spool 24 is contained in the tubular shell body 23a in a rotatable manner, and provided with the photo film 22 wound thereabout. At first, the photo film cassette 16 has a form in which the photo film 22 is wound about the spool 24 and contained in the cassette shell 23 in an initial step of the photo film unit producing line. In order to load the housing 3 with the photo film cassette 16, the photo film 22 is unwound from the cassette shell 23, and handled and wound externally in a roll form, and become contained in the main body 15 in a separate manner from the cassette shell 23.

### Example 1

In Table 12, evaluation of characteristics of a task is indicated in relation to the task of managing failure of occurrence in raw inspection in photo film winding step. A score obtained from the skill categories of learning of the task is 24 points. A score obtained from the frequency is 5 points. A score obtained from available time is 5 points. According to the summation, a total score of those in relation to learning is 34 points. A score in relation to constituents of risk is 9 points. According to the summation, a total score of those in relation to learning and risk is 43 points. For example, if a rule is applied in that a task of a score of 30 points or less should be allocated to employees, the task of 24 points becomes allocated to employees.

TABLE 12
EXAMPLE 1: MANAGING FAILURE OF OCCURRENCE IN
RAW INSPECTION IN PHOTO FILM WINDING STEP

| | | RANKS OF EVALUATION | | | | | |
|---|---|---|---|---|---|---|---|
| FACTORS OF EVALUATION | | R0 | R1 | R2 | R3 | R4 | R5 |
| INPUT: | (a) KNOWLEDGE | — | — | — | — | — | 12a |
| SENSING, RECOGNITION & | (b) COGNITION | — | — | — | — | 12b | — |
| ACQUISITION OF INFO | | | | | | | |
| PROCESS: | (c) KNOWLEDGE | — | — | — | — | — | 12c |
| CONSIDERATION | (d) THINKING | — | — | — | — | 12d | — |
| & DECISION | | | | | | | |
| OUTPUT: | (e) METHOD | — | — | — | 12e | — | — |
| BEHAVIOR & ACTION | (f) COGNITION | — | — | — | 12f | — | — |
| | FOR ACTION | | | | | | |
| (g) FREQUENCY | | — | — | — | — | — | 12g |
| (h) AVAILABLE TIME (ENABLING | | — | — | — | — | — | 12h |
| HANDLING) | | | | | | | |
| RISK OF | (i) PRODUCT | — | — | — | — | — | 12i |
| INFLUENCE | QUALITY | | | | | | |
| OF ERRORS | (j) COST | — | — | — | 12j | — | — |
| | (k) SAFETY | — | 12k | — | — | — | — |
| | (l) | 12l | — | — | — | — | — |
| | ENVIRONMENT | | | | | | |

In the table, reference signs represent the following descriptions.

R0: 0, Promptly learnable

R1: 1, Easy to learn

R2: 2, Possible to learn

R3: 3, Possible to learn with much work

R4: 4, Learnable with possible difficulty

R5: 5, Difficult to learn

12a: Knowledge is required regarding shapes of film scratches of tens of kinds, & influence of such film scratches to final products.

12b: Info of cognition is required regarding angles & positions of objects, light & eyes so as to find film scratches. Patterns of occurrence of scratches are limited in comparison with photo film producing step.

12c: Knowledge is required regarding shapes of film scratches, influence of such film scratches to final products, & estimated causes of scratches in consideration of structures of machinery.

12d: Determination of causal relationships of occurrence of film scratches is required in consideration of shapes of film scratches, & structures of machinery. Patterns of occurrence of scratches are limited in comparison with photo film producing step.

12e: Operation is complex to specify producing lot where film scratches have occurred.

12f: Patterned cognition is a greater amount regarding angles & positions of objects, light & eyes, owing to having found producing lot of occurrence of film scratches.

12g: Frequency of occurrence of film scratches is 1 time or lower per 1 month.

12h: Immediate response is required because scratched products of photo films may flow to next producing sequence & may dispatch.

12i: Failing products may dispatch from factory with middle probability owing to daily schedule of inspection for dispatch.

12j: Possible occurrence of loss of 2 million yen or more due to reloading of photo film.

12k: Possible occurrence of light scratches upon managing failure of photo films.

12l: No fluid or gas in particular flows out of factory.

### Example 2

In Table 13, evaluation of characteristics of a task is indicated in relation to the task of managing a temporary stop of machinery in photo film winding step. A score obtained from the skill categories of learning of the task is 25 points. A score in relation to constituents of risk is 1 point. According to the summation, a total score of those is 26 points. For example, if a rule is applied in that a task of a score of 30 points or less should be allocated to leased workers, the task of 26 points becomes allocated to leased workers. In contrast, if a rule is applied in that a task of a score of 20-30 points should be allocated by decision of those who administer the producing sequence, then the task of 26 points is allocated to a selected one of staff on load and regular employees in a manner depending on a situation.

TABLE 13
EXAMPLE 2: MANAGING TEMPORARY STOP OF MACHINERY
IN PHOTO FILM WINDING STEP

| | | RANKS OF EVALUATION | | | | | |
|---|---|---|---|---|---|---|---|
| FACTORS OF EVALUATION | | R0 | R1 | R2 | R3 | R4 | R5 |
| INPUT: | (a) KNOWLEDGE | — | — | — | 13a | — | — |
| SENSING, | (b) COGNITION | — | — | — | 13b | — | — |
| RECOGNITION & | | | | | | | |
| ACQUISITION OF INFO | | | | | | | |
| PROCESS: | (c) KNOWLEDGE | — | — | — | 13c | — | — |
| CONSIDERATION | (d) THINKING | — | — | 13d | — | — | — |
| & DECISION | | | | | | | |
| OUTPUT: | (e) METHOD | — | — | — | 13e | — | — |
| BEHAVIOR & | (f) COGNITION | — | — | — | 13f | — | — |
| ACTION | FOR ACTION | | | | | | |
| (g) FREQUENCY | | — | — | — | — | 13g | — |
| (h) AVAILABLE TIME (ENABLING | | — | — | — | — | 13h | — |
| HANDLING) | | | | | | | |
| RISK OF | (i) PRODUCT | — | 13i | — | — | — | — |
| INFLUENCE OF | QUALITY | | | | | | |
| ERRORS | (j) COST | — | 13j | — | — | — | — |
| | (k) SAFETY | — | 13k | — | — | — | — |
| | (l) | 13l | — | — | — | — | — |
| | ENVIRONMENT | | | | | | |

In the table, reference signs represent the following descriptions.

For R0-R5 the descriptions in Table 12 are repeated.

13a: Knowledge is required for normal & abnormal states of electromagnetic valve, suction hose, suction pad & the like which may break frequently.

13b: Cognition is required as to sound of opening & closing of valve, its timing, & usual & unusual states of the touch of pad.

13c: Limited knowledge is required for the entire machinery for discriminating the states of electromagnetic valve & suction hose, in a form of considerably patterned knowledge.

13d: Causes of abnormality of the valve & hose are known as very patterned structure of problems.

13e: Exchange of the electromagnetic valve & suction pad requires recovery of complicated steps depending on situations.

13f: Info of positioning parts is a factor of cognition within a task of repetition, such as exchange of parts.

13g: 1 time or so per 2 weeks.

13h: Recovery as early as possible is required as breakage may influence proportion of system operation.

13i: Only influence within producing sequence, as suitable recovery is required for restarting machinery.

13j: Loss is only 100,000 yen or less even if time for recovery is extended slightly.

13k: Light scratches are likely to occur upon managing breakage of the machinery.

13l: No fluid or gas in particular flows out of factory.

### Example 3

In Table 14, evaluation of characteristics of a task is indicated in relation to filling in of a log of record and summation in photo film winding step. A score obtained from the skill categories of learning of the task is 5 points. A score in relation to constituents or risk is 0 point. According to the summation, a total score of those is 5 points. For example, if a rule is applied in that a task of a score of 30 points or less should be allocated to leased workers, the task of 5 points becomes allocated to leased workers.

TABLE 14
EXAMPLE 4: MANAGING FAILURE UPON STOP OF MACHINERY
IN PHOTO FILM LOADING STEP

| | | RANKS OF EVALUATION | | | | | |
|---|---|---|---|---|---|---|---|
| FACTORS OF EVALUATION | | R0 | R1 | R2 | R3 | R4 | R5 |
| INPUT: | (a) KNOWLEDGE | 14a | — | — | — | — | — |
| SENSING, RECOGNITION & | (b) COGNITION | 14b | — | — | — | — | — |
| ACQUISITION OF INFO | | | | | | | |
| PROCESS: | (c) KNOWLEDGE | — | 14c | — | — | — | — |
| CONSIDERATION | (d) THINKING | — | 14d | — | — | — | — |
| & DECISION | | | | | | | |
| OUTPUT: | (e) METHOD | 14e | — | — | — | — | — |
| BEHAVIOR & ACTION | (f) COGNITION | 14f | — | — | — | — | — |
| | FOR ACTION | | | | | | |
| (g) FREQUENCY | | — | — | 14g | — | — | — |
| (h) AVAILABLE TIME (ENABLING | | — | 14h | — | — | — | — |
| HANDLING) | | | | | | | |
| RISK OF | (i) PRODUCT | 14i | — | — | — | — | — |
| INFLUENCE OF | QUALITY | | | | | | |
| ERRORS | (j) COST | 14j | — | — | — | — | — |
| | (k) SAFETY | 14k | — | — | — | — | — |
| | (l) | 14l | — | — | — | — | — |
| | ENVIRONMENT | | | | | | |

In the table, reference signs represent the following descriptions.

For R0-R5 the descriptions in Table 12 are repeated.

14a: Only knowledge of modes of stop of machinery, cleanup, & periodical inspection is required.

14b: No specialized cognition.

14c: Knowledge of time & way of stop is required.

14d: Simple thinking pattern is required for filling in log of record.

14e: Skill of writing normally is sufficient.

14f: Only behavior of writing normally is sufficient.

14g: 1 time per 1 shift of shift work of worker.

14h: Sufficient time is available in view of task but performing is required before next shift of shift work.

14i: No direct influence to product quality.

14j: No influence to cost even upon errors in filling in.

14k: No cause of problem in safety.

14l: No fluid or gas in particular flows out of factory.

### Example 4

Table 15 indicates results of evaluation of tasks of managing failure upon stop of machinery in photo film loading step, where the cassette shell 23 is loaded with the photo film 22 in the producing line of the photo film cassette or cartridge 16 of FIG. 12.

TABLE 15
EXAMPLE 4: MANAGING FAILURE UPON STOP OF MACHINERY
IN PHOTO FILM LOADING STEP

| | | RANKS OF EVALUATION | | | | | |
|---|---|---|---|---|---|---|---|
| FACTORS OF EVALUATION | | R0 | R1 | R2 | R3 | R4 | R5 |
| INPUT: | (a) KNOWLEDGE | — | — | — | — | 15a | — |
| SENSING, RECOGNITION & | (b) COGNITION | — | — | — | — | 15b | — |
| ACQUISITION OF INFO | | | | | | | |
| PROCESS: | (c) KNOWLEDGE | — | — | — | — | 15c | — |
| CONSIDERATION & DECISION | (d) THINKING | — | — | — | 15d | — | — |
| OUTPUT: | (e) METHOD | — | — | — | 15e | — | — |
| BEHAVIOR & | (f) COGNITION | — | — | — | — | 15f | — |
| ACTION | FOR ACTION | | | | | | |
| (g) FREQUENCY | | — | — | — | — | 15g | — |
| (h) AVAILABLE TIME (ENABLING HANDLING) | | — | — | — | — | 15h | — |
| RISK OF | (i) PRODUCT | — | 15i | — | — | — | — |
| INFLUENCE OF | QUALITY | | | | | | |
| ERRORS | (j) COST | — | 15j | — | — | — | — |
| | (k) SAFETY | — | 15k | — | — | — | — |
| | (l) | 15l | — | — | — | — | — |
| | ENVIRONMENT | | | | | | |

In the table, reference signs represent the following descriptions.

For R0-R5 the descriptions in Table 12 are repeated.

15a: Correctly acquired knowledge is required regarding sensors informing failure, & positions of disposing the sensors.

15b: Info of cognition is required regarding sound of machinery & physical touch of objects because breakage actually occurs in the darkroom.

15c: Correctly acquired knowledge is required regarding names of sensors, & relevant features of breakage, also with technical knowledge.

15d: Mixture of patterned & unpatterned thinking is usual for the breakage of machinery.

15e: Recovery of breakage is a slightly complicated step due to mixture of patterned & unpatterned action.

15f: Learning of cognition needs by spending learning time for layout of the machinery & indoor spaces on upper, lower & lateral sides.

15g: 1 time or so per 2 weeks.

15h: Recovery as early as possible is required as breakage may influence productivity.

15i: Only influence within producing sequence, as suitable recovery is required for restarting machinery.

15j: Loss is only 100,000 yen or less even if time for recovery is extended slightly.

15k: Light scratches are likely to occur upon managing breakage of the machinery.

15l: No fluid or gas in particular flows out of factory.

Automated machinery is used in the photo film loading step, and installed in a darkroom. There is an indexing table for winding, where the spool 24 is supported, and a leading end of photo film is inserted into a connection hole in the spool 24 with the photo film in a strip having a regular length. The indexing table rotates the spool 24 and winds the photo film. Then the spool 24 is inserted into the tubular shell body 23a with the photo film 22, before the shell caps 23b are fitted on ends of the tubular shell body 23a. Tens of sensors are disposed in the step for detecting failure. A worker or operator is not in the darkroom, but stands at a console in an illuminated room, and checks a status of the working. When the sensors detect failure, results of the detection are indicated on the console. The worker enters the darkroom if required according to the failure.

It is observed that a high score is determined in relation to the difficulty level, frequency (g) and available time (h) for the task. A reason of the high score for the difficulty level is that reading of results of outputs of the sensor on the display of the console is difficult and needs high skill to find the meaning of failure. Also, a manual task must be performed in the darkroom as environment. As described for the KNOWLEDGE factor (a) of the INPUT skill category, correctly acquired knowledge is required regarding sensors informing failure, and positions of disposing the sensors in the darkroom, for the purpose of rapid working in the darkroom.

To modify the task to enable learning in a reduced term, it is possible in FIG. 9 to apply the item of Clearly illustrate present state of system, and the item of Record info externally except for memory, under the section of Utilize similarity to reduce use of memory. Specifically, a display panel of a console for results of sensors can be used, and driven to indicate normal or abnormal states of the inside of the darkroom for virtual checking of positions of occurrence. Therefore, the task is simplified regarding quality and quantity of knowledge of a worker. His or her required minimum knowledge decreases suitably for checking errors. In Table 16, the score of the factors of the KNOWLEDGE in the INPUT skill category can be reduced from 4 points to 2 points.

TABLE 16
EXAMPLE 4: MANAGING FAILURE UPON STOP OF MACHINERY IN PHOTO
FILM LOADING STEP (RE-EVALUATION AFTER IMPROVEMENT)

| | | RANKS OF EVALUATION | | | | | |
|---|---|---|---|---|---|---|---|
| FACTORS OF EVALUATION | | R0 | R1 | R2 | R3 | R4 | R5 |
| INPUT: | (a) KNOWLEDGE | — | — | 16a | <- | <- x | — |
| SENSING, RECOGNITION & | (b) COGNITION | — | — | — | — | 16b | — |
| ACQUISITION OF INFO | | | | | | | |
| PROCESS: | (c) KNOWLEDGE | — | — | — | 16c | <- x | — |
| CONSIDERATION | (d) THINKING | — | — | — | 16d | — | — |
| & DECISION | | | | | | | |
| OUTPUT: | (e) METHOD | — | — | — | 16e | — | — |
| BEHAVIOR & ACTION | (f) COGNITION | — | — | 16f | <- | <- x | — |
| | FOR ACTION | | | | | | |
| (g) FREQUENCY | | — | — | — | — | 16g | — |
| (h) AVAILABLE TIME (ENABLING | | — | — | — | — | 16h | — |
| HANDLING) | | | | | | | |
| RISK OF | (i) PRODUCT | — | 16i | — | — | — | — |
| INFLUENCE OF | QUALITY | | | | | | |
| ERRORS | (j) COST | — | 16j | — | — | — | — |
| | (k) SAFETY | — | 16k | — | — | — | — |
| | (l) | 16l | — | — | — | — | — |
| | ENVIRONMENT | | | | | | |

In the table, reference signs represent the following descriptions.

For R0-R5 the descriptions in Table 12 are repeated.

16a: Positions of occurrence & normal & abnormal present states in the darkroom are viewed on display virtually, to enable recognition only with small previous knowledge.

16b: Owing to machinery in the darkroom, actually broken states are found by aurally finding sound of the machinery, or physically touching object.

16c: Knowledge previously required can be reduced because status can be recognized by use of displayed info.

16d: Mixture of patterned & unpatterned thinking is usual for the breakage of machinery.

16e: Recovery of breakage is a slightly complicated step due to mixture of patterned & unpatterned action.

16f: Spatial perception in the darkroom can be learned with a few times of experience & by use of layout view & current real structure of machinery.

16g: 1 time or so per 2 weeks.

16h: Recovery as early as possible is required as breakage may influence productivity.

16i: Only influence within producing sequence, as suitable recovery is required for restarting machinery.

16j: Loss is only 100,000 yen or less even if time for recovery is extended slightly.

16k: Light scratches are likely to occur upon managing breakage of the machinery.

16l: No fluid or gas in particular flows out of factory.

On the basis of improvement in the KNOWLEDGE factor (a) of the INPUT skill category, the scores of the KNOWLEDGE factor (c) of the PROCESS skill category, and the COGNITION-FOR-ACTION factor (f) of the OUTPUT skill category are decreased too. The score of the evaluation of the characteristics can be reduced from 33 points to 28 points. In addition, simulated experience of tasks can be effectively increased by utilizing task learning tools or the like. The score can be reduced in a further manner. It is possible to allocate the task to leased workers as well as regular employees according to the decrease in the score of evaluating the characteristics by the improvement. This is effective in achieving low-cost operation.

It is possible in working of the invention to systematize difficulty levels of tasks as standards in a commonly available manner. Improvement of tasks can be specifically effected. For example, a manual or handbook can be created to enable the short term learning, so as to foster workers with skill by use of the manual. Also, difficulty can be lowered by improving the machinery. Furthermore, a structure of causes of workers in an associated producing sequence can be suitably modified. As applications of the present invention, task allocation may be applied not only in the producing sequence but in a division of the producing line, or in the entirety of the factory. Also, a manual for the short term learning and a tool of learning can be improved effectively. The improvement of the machinery can be systematized. A low cost operation can developed in the factory field, so results of the improvement can be verified at one time.

In the above embodiment, the numbers of factors are determined with six ranks for difficulty levels, one factor for frequency, one factor for available time, and four criteria for influence level of predictable errors. However, those numbers may be modified in any suitable manner in the invention. The numbers of skill categories and criteria are determined with six skill categories of learning, and six criteria for influence level of errors as constituents of risk. However, those numbers may be modified in any suitable manner. Furthermore, the measures, modifications and the like for the improvement of the task are not limited to the above embodiment, but can be determined in any suitable manner in compliance with various kinds of tasks. In the above embodiment, the producing line produces the lens fitted photo film unit with the photo film cassette. However, a producing line of the invention may be any producing system for industrially producing a product.

Also, it is possible to use a computer to evaluate characteristics of a task. For use with the computer, a storage device is connected for storing various sets of information in forms of tables, such information including types of the producing line, producing sequences and the tasks, specific information of evaluating the characteristics of the tasks, grouping of the tasks of the span of management and the tasks of other categories, definitions of factors of difficulty, frequency, available time and the like as skill categories of the learning, definitions of the constituents of risk in influence levels of predictable errors and coefficients of modeled occurrence of errors, and ranks, measures and score of the factors. Software can be combined with a computer for execution of programs related to various functions, which are a function to input specific information of the task, a function to evaluate the characteristics of the task by referring to stored tables according to the input specific information, a function to allocate the task between employees and leased workers according to the score, a function to generate information of a list of improving items of the task according to the score, and a function to simulate improvement for the purpose of reducing the score or reducing difficulty. Also, skills of working of workers may be evaluated as well as the characteristics of the tasks, and utilized for determining allocation of the workers to the tasks.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A difficulty evaluating method of evaluating a task difficulty level of a task within a producing sequence, said difficulty evaluating method comprising steps of:
categorizing a skill required for human resource to perform said task according to cognitive science to determine at least first, second and third skill categories, said first skill category being related to cognition of outer field information or acquisition of cognitive information, said second skill category being related to thinking or decision, and said third skill category being related to behavior or action;
evaluating said task with said first, second and third skill categories, to obtain respectively difficulty ranks; and
determining said task difficulty level by processing said difficulty ranks in combination according to overall processing.

2. A difficulty evaluating method as defined in claim 1, wherein plural factors are provided in each of said first, second and third skill categories;.
factors of said first skill category among said factors have an evaluating factor of knowledge with sufficient quality or quantity for correctly recognizing a status, and an evaluating factor of cognition with degree of necessity of sensory organs for correctly recognizing a status;
factors of said second skill category among said factors have an evaluating factor of knowledge with sufficient quality or quantity for correctly deciding, and an evaluating factor of thinking with complexity in steps of decision, or large quantity of factors of decision;
factors of said third skill category among said factors have an evaluating factor of performing with complexity or variety of methods or measures associated with said task, and an evaluating factor of cognition for difficulty in repetition of action for correct work.

3. A risk diagnosing method of diagnosing risk of a task within a producing sequence, said risk diagnosing method comprising steps of:
predetermining plural assessing criteria including a product quality criterion, a cost loss criterion, a safety criterion, and an environmental criterion, for assessment of a predictable error of said task;
assessing said task with said plural assessing criteria, to obtain respectively influence ranks; and
determining an error influence level of said error by processing said influence ranks in combination according to overall processing, influence of said error influence level being related to said producing sequence or a producing line having said producing sequence.

4. A risk diagnosing method as defined in claim 3, wherein said product quality criterion is based on estimated influence to product quality upon occurrence of said error;
said cost loss criterion is based on an estimated financial loss caused upon occurrence of said error;
said safety criterion is based on estimated danger of said task in said error; and
said environmental criterion is based on estimated influence to environment upon occurrence of said error.

5. A characteristic evaluating method of evaluating a characteristic of a task within a producing sequence, said characteristic evaluating method comprising steps of:
determining a constituent of learning in relation to difficulty in learning said task;
determining a constituent of risk in relation to influence of a predictable error of said task to said producing sequence or a producing line having said producing sequence; and
determining said characteristic of said task by processing said constituent of learning and said constituent of risk in combination according to overall processing.

6. A characteristic evaluating method as defined in claim 5, wherein said plurality of said task is classified into a first task related to a range equal to or greater than a range of said producing sequence, and a second task related to installed machinery in said producing sequence, and said second task is subjected to evaluating of said characteristic.

7. A characteristic evaluating method as defined in claim 5, wherein said constituent of learning is evaluated by combined evaluation of a difficulty level of difficulty of said task, frequency of occurrence of said task, and constraint of available time required for said task.

8. A characteristic evaluating method as defined in claim 7, wherein said difficulty level is determined by steps including:
categorizing a skill required for human resource to perform said task according to cognitive science to determine at least first, second and third skill categories, said first skill category being related to cognition of outer field information or acquisition of cognitive information, said second skill category being related to thinking or decision, and said third skill category being related to behavior or action;
evaluating said task with said first, second and third skill categories, to obtain respectively difficulty ranks, said difficulty ranks being processed in combination according to overall processing for difficulty level determination.

9. A characteristic evaluating method as defined in claim 8, wherein plural factors are provided in each of said first, second and third skill categories;
factors of said first skill category among said factors have an evaluating factor of knowledge with sufficient quality or quantity for correctly recognizing a status, and an evaluating factor of cognition with degree of necessity of sensory organs for correctly recognizing a status;
factors of said second skill category among said factors have an evaluating factor of knowledge with sufficient quality or quantity for correctly deciding, and an evaluating factor of thinking with complexity in steps of decision, or large quantity of factors of decision;
factors of said third skill category among said factors have an evaluating factor of performing with complexity or variety of methods or measures associated with said task, and an evaluating factor of cognition for difficulty in repetition of action for correct work.

10. A characteristic evaluating method as defined in claim 7, wherein said constituent of risk is corrected according to a coefficient of modeled error occurrence determined according to said difficulty level.

11. A characteristic evaluating method as defined in claim 5, wherein said constituent of risk is determined by steps including:
predetermining plural assessing criteria including a product quality criterion, a cost loss criterion, a safety criterion, and an environmental criterion, for error assessment of said task;
assessing said task with said plural assessing criteria, to obtain respectively influence ranks, said constituent of risk being determined by processing to overall processing said influence ranks in combination according to overall processing.

12. A characteristic evaluating method as defined in claim 11, wherein said product quality criterion is based on estimated influence to product quality upon occurrence of said error;
said cost loss criterion is based on an estimated financial loss caused upon occurrence of said error;
said safety criterion is based on estimated danger of said task in said error; and
said environmental criterion is based on estimated influence to environment upon occurrence of said error.

13. A task allocating method of allocating a task within a producing sequence, said task allocating method comprising steps of:
determining a constituent of learning in relation to difficulty in learning said task;
determining a constituent of risk in relation to influence of a predictable error of said task to said producing sequence or a producing line having said producing sequence;
determining a characteristic of said task by processing said constituent of learning and said constituent of risk in combination according to overall processing; and
allocating said task to human resource in consideration of said characteristic of said task.

14. A task allocating method as defined in claim 13, wherein said human resource includes persons between which a degree of responsibility or duty is different.

15. A task improving method of improving a task within a producing sequence, said task improving method comprising steps of:
determining a constituent of learning in relation to difficulty in learning said task;
determining a constituent of risk in relation to influence of a predictable error of said task to said producing sequence or a producing line having said producing sequence;
determining a characteristic of said task by processing said constituent of learning and said constituent of risk in combination according to overall processing; and
improving said task in consideration of said characteristic of said task.

16. A task allocating method as defined in claim 15, wherein after said improving step, a characteristic of said task is evaluated again to check an improved state.

17. A task allocating method of allocating a task within a producing sequence, said task allocating method comprising steps of:
categorizing a skill required for human resource to perform said task according to cognitive science to determine at least first, second and third skill categories, said first skill category being related to cognition of outer field information or acquisition of cognitive information, said second skill category being related to thinking or decision, and said third skill category being related to behavior or action;
evaluating said task with said first, second and third skill categories, to obtain respectively difficulty ranks;
determining said task difficulty level by processing said difficulty ranks in combination according to overall processing; and
allocating said task to human resource in consideration of said characteristic of said task.

18. A task allocating method of allocating a task within a producing sequence, said task allocating method comprising steps of:
predetermining plural assessing criteria including a product quality criterion, a cost loss criterion, a safety criterion, and an environmental criterion, for assessment of a predictable error of said task;
assessing said task with said plural assessing criteria, to obtain respectively influence ranks;
determining an error influence level of said error by processing said influence ranks in combination according to overall processing, influence of said error influence level being related to said producing sequence or a producing line having said producing sequence; and
allocating said task to human resource in consideration of said characteristic of said task.
